(19) United States
(12) Patent Application Publication
(10) Pub. No.: US 2026/0257978 A1
(43) Pub. Date: Sep. 3, 2026

(54) DESTRUCTION OF PERFLUORO- OR POLYFLUORO-ALKYL SUBSTANCES

(71) Applicants: Carlos Borras, Ruskin, FL (US); Javier Brito, Lakeland, FL (US); Donald A. Luke, Valrico, FL (US); Kyle Broaddrick, Brookfield, WI (US)

(72) Inventors: Carlos Borras, Ruskin, FL (US); Javier Brito, Lakeland, FL (US); Donald A. Luke, Valrico, FL (US); Kyle Broaddrick, Brookfield, WI (US)

(21) Appl. No.: 19/656,864

(22) Filed: Apr. 23, 2026

Related U.S. Application Data
(63) Continuation of application No. 19/444,084, filed on Jan. 8, 2026.
Application No. 19/444,084 is a continuation-in-part of application No. PCT/US2025/036696, filed on Jul. 7, 2025.
(60) Provisional application No. 63/668,476, filed on Jul. 8, 2024.
(60) Provisional application No. 63/795,011, filed on Apr. 25, 2025.

(51) Int. Cl. C07C 17/42; C07C 17/389

(52) U.S. Cl. CPC C07C 17/42; C07C 17/389

(57) ABSTRACT

A method of destroying one or more perfluoro- or polyfluoro-alkyl substances (PFAS) includes performing a destruction procedure on an aqueous composition to form a treated aqueous composition. The aqueous composition includes the one or more PFAS. The treated aqueous composition has a concentration of the one or more PFAS that is lower than a concentration of the one or more PFAS in the aqueous composition. The method includes aerating the treated aqueous composition to form a foam thereon. The method also includes separating the foam from the treated aqueous composition to form a concentrate and a clarified aqueous composition. The clarified aqueous composition has a concentration of the one or more PFAS that is lower than a concentration of the one or more PFAS in the concentrate.

### CROSS-REFERENCE TO RELATED APPLICATIONS

This application claims the benefit of priority to U.S. Provisional Patent Application Ser. No. 63/795,011 filed Apr. 25, 2025, and to U.S. Utility application Ser. No. 19/444,084 filed Jan. 8, 2026, which is a continuation-in-part of and claims the benefit of priority under 35 U.S.C. § 120 to PCT Application PCT/US2025/036696 filed Jul. 7, 2025, which claims the benefit under 35 U.S.C. § 119 (e) of U.S. Provisional Patent Application Ser. No. 63/668,476 filed Jul. 8, 2024, the disclosures of which are incorporated herein in their entirety by reference.

### BACKGROUND

Perfluoroalkyl or polyfluoroalkyl substances (PFAS) embody a range of polyfluorinated alkyl substances including but not limited to carboxylic acids, alkyl sulfonates, alkyl sulfonamide compounds, and fluorotelemer compounds of differing carbon chain lengths and precursors thereof. PFAS have found use in a wide variety of applications including as a specialized fire-fighting product, or for impregnation or coating of textiles, leather and carpet, or for carpet cleaning compounds, as well as in aviation hydraulic fluids, metal plating, agricultural (e.g., insect traps for certain types of ants), photo-imaging, electronics manufacture, and non-stick cookware applications.

PFAS include but are not limited to perfluorooctane sulfonate (PFOS), perfluorooctanoic acid (PFOA) and perfluorohexane sulfonate (PFHxS), which are compounds of concern that are resistant to biotic or abiotic degradation and thus are persistent in the environment. They are recalcitrant, bio-accumulative, and known to contaminate soils, groundwaters, and drinking water supplies. PFOS, PFHxS, and PFOA have published human health and environmental regulatory criteria in most developed world jurisdictions. Additional PFAS are expected to be identified as contaminants of concern as new research toxicology data indicates potential risk associations.

Conventional techniques for treatment of water to remove and/or destroy PFAS suffer from problems of inefficiency, ineffectiveness, lack of versatility, high expense, and complex implementation.

### SUMMARY OF THE INVENTION

Various aspects of the present disclosure provide a method of destroying one or more perfluoro- or polyfluoro-alkyl substances (PFAS). The method includes performing a destruction procedure on an aqueous composition to form a treated aqueous composition. The aqueous composition includes the one or more PFAS. The treated aqueous composition has a concentration of the one or more PFAS that is lower than a concentration of the one or more PFAS in the aqueous composition. The method includes aerating the treated aqueous composition to form a foam thereon. The method also includes separating the foam from the treated aqueous composition to form a concentrate and a clarified aqueous composition, wherein the clarified aqueous composition has a concentration of the one or more PFAS that is lower than a concentration of the one or more PFAS in the concentrate.

Various aspects of the present disclosure provide a method of destroying one or more perfluoro- or polyfluoro-alkyl substances (PFAS). The method includes electrolytically treating an aqueous composition in an electrochemical cell. The aqueous composition has a pH of 0 to 5.5 and includes the one or more PFAS. The electrolytic treatment of the aqueous composition in the electrochemical cell forms an electrolytically-treated aqueous composition. The electrolytically-treated aqueous composition has a concentration of the one or more PFAS that is lower than a concentration of the one or more PFAS in the aqueous composition. The method includes aerating the electrolytically-treated aqueous composition to form a foam thereon. The method also includes separating the foam from the electrolytically-treated aqueous composition. The electrolytically-treated aqueous composition separated from the foam has a concentration of the one or more PFAS that is lower than a concentration of the one or more PFAS in the separated foam.

Various aspects of the present disclosure provide a method of destroying one or more perfluoro- or polyfluoro-alkyl substances (PFAS). The method includes electrolytically treating an aqueous composition in an electrochemical cell. The aqueous composition has a pH of 1 to 3.5 and includes the one or more PFAS and a metal component. The electrolytic treatment of the aqueous composition in the electrochemical cell forms an electrolytically-treated aqueous composition. The electrolytically-treated aqueous composition has a concentration of the one or more PFAS that is lower than a concentration of the one or more PFAS in the aqueous composition. The method includes aerating the electrolytically-treated aqueous composition to form a foam thereon. The method includes separating the foam from the electrolytically-treated aqueous composition to form a concentrate and a clarified aqueous composition. The clarified aqueous composition has a concentration of the one or more PFAS that is lower than a concentration of the one or more PFAS in the concentrate. The method also includes electrolytically treating the concentrate in the electrochemical cell.

Various aspects of the present disclosure provide a method of destroying one or more perfluoro- or polyfluoro-alkyl substances (PFAS). The method includes electrolytically treating an aqueous composition in an electrochemical cell, wherein the aqueous composition has a pH of 0 to 5.5 and includes aluminum hydroxide and the one or more PFAS, to form an electrolytically-treated aqueous composition. The electrolytically-treated aqueous composition has a concentration of the one or more PFAS that is 0.0001% to 90% of a concentration of the one or more PFAS in the aqueous composition. The method includes aerating the electrolytically-treated aqueous composition to form a foam thereon. The method includes separating the foam from the electrolytically-treated aqueous composition. The electrolytically-treated aqueous composition separated from the foam has a concentration of the one or more PFAS that is 0.0001% to 50% of a concentration of the one or more PFAS in the separated foam. The method also includes electrolytically treating the separated foam in the electrochemical cell to form a second electrolytically-treated aqueous composition that has a concentration of the one or more PFAS that is 0.0001% to 90% of a concentration of the one or more PFAS in the separated foam.

Various aspects of the present disclosure provide a method of destroying one or more perfluoro- or polyfluoro-alkyl substances (PFAS). The method includes electrolytically treating an aqueous composition in an electrochemical cell, wherein the aqueous composition has a pH of 0 to 5.5 and includes aluminum hydroxide and the one or more PFAS, and monitoring the concentration of the one or more PFAS in the aqueous composition during the electrolytic treatment of the aqueous composition and continuing to perform the electrolytic treatment until the concentration of the one or more PFAS in the aqueous composition reaches a predetermined concentration, to form an electrolytically-treated aqueous composition. The electrolytically-treated aqueous composition has a concentration of the one or more PFAS that is 0.0001% to 90% of a concentration of the one or more PFAS in the aqueous composition. The method includes aerating the electrolytically-treated aqueous composition to form a foam thereon. The method also includes separating the foam from the electrolytically-treated aqueous composition, wherein the electrolytically-treated aqueous composition separated from the foam has a concentration of the one or more PFAS that is 0.0001% to 50% of a concentration of the one or more PFAS in the separated foam.

Various aspects of the present disclosure provide a method of destroying one or more perfluoro- or polyfluoro-alkyl substances (PFAS). The method includes performing a destruction procedure on an aqueous composition to form a treated aqueous composition. The aqueous composition includes the one or more PFAS and includes an additive including a metal component that includes a metal. The treated aqueous composition has a concentration of the one or more PFAS that is lower than a concentration of the one or more PFAS in the aqueous composition. The method includes aerating the treated aqueous composition to form a foam thereon. The method includes removing the foam from the treated aqueous composition to form a concentrate including the fluoroalkyl compound and to form a clarified aqueous composition including the metal of the metal component of the additive. A ratio of a concentration of the fluoroalkyl compound in the aqueous composition to a concentration of the fluoroalkyl compound in the concentrate is 1:2 to 1:2,000,000. The method includes performing the destruction procedure on the concentrate. The method includes reusing the metal from the clarified aqueous composition or an extract thereof. The reusing includes adding the metal to the treated aqueous composition of a subsequent iteration of the method.

Various aspects of the present disclosure provide a method of destroying one or more perfluoro- or polyfluoro-alkyl substances (PFAS). The method includes performing a destruction procedure on an aqueous composition to form a treated aqueous composition. The aqueous composition includes the one or more PFAS, has a pH of 0 to 5.5, and includes an additive including a metal component that includes aluminum. The treated aqueous composition has a concentration of the one or more PFAS that is lower than a concentration of the one or more PFAS in the aqueous composition. The method includes aerating the treated aqueous composition to form a foam thereon. The method includes removing the foam from the aqueous composition, wherein the aqueous composition having had the foam removed therefrom is a second aqueous composition. The method includes aerating the second aqueous composition to form a second foam and removing the second foam from the second aqueous composition, and combining the foam removed from the aqueous composition and the second foam to form a concentrate including the fluoroalkyl compound. The second aqueous composition having the second foam removed therefrom is a clarified aqueous composition including the aluminum of the metal component. The method includes reusing the aluminum in the clarified aqueous composition or an extract thereof. The reusing includes adding the aluminum to the treated aqueous composition. The method includes aerating the concentrate to form a secondary foam including the fluoroalkyl compound then removing the secondary foam from the concentrate to form a secondary concentrate including the fluoroalkyl compound and to form a secondary clarified aqueous composition, wherein during the formation of the secondary foam the concentrate has a pH of 0 to less than 4, and wherein a ratio of a concentration of the fluoroalkyl compound in the treated aqueous composition to a concentration of the fluoroalkyl compound in the secondary concentrate is 1:2 to 1:2,000,000. The method includes performing the destruction procedure on the secondary concentrate. The method also includes recycling the secondary clarified aqueous composition back to or upstream of the treated aqueous composition.

Various aspects of the present disclosure have advantages over other methods of destroying PFAS in water. For example, various aspects of the present disclosure address limitations of conventional approaches that apply a destruction procedure to an aqueous composition containing PFAS at dilute concentrations, which can result in longer treatment durations, greater energy consumption per unit of PFAS destroyed, and diminishing returns as the PFAS concentration in the aqueous composition decreases during the destruction procedure. Various aspects of the present disclosure address limitations of conventional approaches that rely on a single destruction step to reduce PFAS concentrations in an aqueous composition to target levels, which can require extended treatment times and can be accompanied by reduced destruction rates as the PFAS concentration in the aqueous composition declines. Advantages can include destruction efficiency advantages, energy consumption advantages, treatment time advantages, process flexibility advantages, reagent reuse advantages, byproduct management advantages, or a combination thereof.

Various aspects of the method of the present disclosure provide destruction efficiency and energy consumption advantages over conventional approaches to destroying PFAS in an aqueous composition. For example, various aspects of the present disclosure perform the destruction procedure on the aqueous composition and then aerate the treated aqueous composition to form a foam in which residual PFAS that were not destroyed during the destruction procedure are concentrated, such that the residual PFAS can be separated from the treated aqueous composition in a concentrate having a PFAS concentration that is greater than the PFAS concentration in the treated aqueous composition. Various aspects of the present disclosure then subject the concentrate to the destruction procedure in a subsequent iteration, such that the residual PFAS are destroyed at a higher concentration at which the destruction procedure proceeds at a higher rate per unit of PFAS destroyed and at a lower energy consumption per unit of PFAS destroyed compared to conventional approaches that continue to operate the destruction procedure on the aqueous composition as the PFAS concentration declines. Various aspects of the present disclosure avoid subjecting aqueous compositions having dilute PFAS concentrations to the destruction procedure, such that the energy and time that would otherwise be expended on destroying PFAS at concentrations at which the destruction procedure has reduced efficiency can be conserved. Various aspects of the present disclosure provide a clarified aqueous composition having a low PFAS concentration more efficiently than conventional approaches that rely on the destruction procedure alone to reduce the PFAS concentration to the same level, because a portion of the PFAS reduction is accomplished by the aeration and foam separation steps rather than by the destruction procedure.

Various aspects of the method of the present disclosure provide treatment time and process flexibility advantages over conventional approaches. For example, various aspects of the present disclosure include performing the destruction procedure, the aeration, and the foam separation, such that the combination of steps can reduce PFAS concentrations in the clarified aqueous composition to target levels in a shorter duration compared to conventional approaches that rely on a single destruction step. Various aspects of the present disclosure include performing multiple iterations of aeration and foam separation, such as sequential foaming of the treated aqueous composition and secondary foaming of the concentrate, such that the degree of PFAS concentration into the foam can be increased and the volume of liquid subjected to the subsequent iteration of the destruction procedure can be reduced. Various aspects of the present disclosure accommodate aqueous compositions obtained from different sources, including water from natural sources, extracts from contaminated soil, extracts from contaminated landfill materials, water contaminated with residual fire-fighting foam, industrial wastewater, or from concentrates prepared from any one of the preceding, and are compatible with aqueous compositions having a range of initial PFAS concentrations. Various aspects of the present disclosure are compatible with different destruction procedures, including thermal treatment, treatment with an electrolytic cell, treatment with a plasma reactor, treatment with supercritical water, combustion treatment, oxidation treatment, and chemical treatment, such that the method of the present disclosure can be combined with destruction technologies selected based on preexisting equipment, preference for a particular destruction process, or the particular PFAS and aqueous composition being treated.

Various aspects of the present disclosure provide reagent reuse and byproduct management advantages over conventional approaches. For example, various aspects of the present disclosure include a metal component such as aluminum hydroxide in the aqueous composition, and the metal component can be recovered from the clarified aqueous composition and reused in a subsequent iteration of the method, such that reagent consumption can be reduced compared to conventional approaches that do not provide for reagent recovery. Various aspects of the present disclosure include purging acidification contaminants such as sulfate and chloride from a recycle stream containing the metal component prior to reuse, such that accumulation of contaminants in the recycled reagent can be managed. Various aspects of the present disclosure include raising the pH of the clarified aqueous composition to precipitate (e.g., further precipitate) the metal component and separating the precipitated metal component from the pH-adjusted aqueous composition, such that the metal component can be recovered in a form suitable for reuse and the separated aqueous composition can be substantially free of the metal component. Various aspects of the present disclosure perform the destruction procedure at low pH and include water traps containing basic water in the headspace of the destruction apparatus, such that volatile PFAS compounds that are released during the destruction procedure can be captured and returned to the process rather than being released to the ambient environment.

### BRIEF DESCRIPTION OF THE FIGURES

The drawings illustrate generally, by way of example, but not by way of limitation, various aspects of the present disclosure.

FIG. 1 illustrates an electrolytic cell, in accordance with various aspects of the present disclosure.

FIG. 2 illustrates two electrolytic cells, in accordance with various aspects of the present disclosure.

FIG. 3 illustrates two electrolytic cells, in accordance with various aspects of the present disclosure.

FIG. 4 illustrates a plurality of electrolytic cells, in accordance with various aspects of the present disclosure.

FIG. 5 illustrates an apparatus for performing a method of concentrating a fluoroalkyl compound, in accordance with various aspects of the present disclosure.

FIG. 6 illustrates a simplified process flow diagram of an apparatus used for destruction and foam separation of PFAS, in accordance with various aspects of the present disclosure.

FIG. 7 illustrates a detailed process flow diagram of an apparatus used for destruction and foam separation of PFAS, in accordance with various aspects of the present disclosure.

FIG. 8 illustrates a graph showing effluent concentrations of various PFAS after foam separation, in accordance with various aspects of the present disclosure.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Concentrations of PFAS in water can be measured by US-EPA methods 533, 537, 537.1, and/or 1633.

### Method of Destroying One or More Perfluoro- or Polyfluoro-Alkyl Substances (PFAS).

Various aspects of the present invention provide a method of destroying one or more perfluoro- or polyfluoro-alkyl substances (PFAS). The method can include performing a destruction procedure on an aqueous composition to form a treated aqueous composition. The aqueous composition includes the one or more PFAS. The treated aqueous composition has a concentration of the one or more PFAS that is lower than a concentration of the one or more PFAS in the aqueous composition. The method can include aerating the treated aqueous composition (e.g., bubbling a gas therethrough) to form a foam thereon. The method can include separating the foam from the treated aqueous composition to form a concentrate and a clarified aqueous composition. The clarified aqueous composition corresponds to the treated aqueous composition having the foam removed therefrom, and the concentrate corresponds to the foam. The clarified aqueous composition has a concentration of the one or more PFAS that is lower than a concentration of the one or more PFAS in the concentrate.

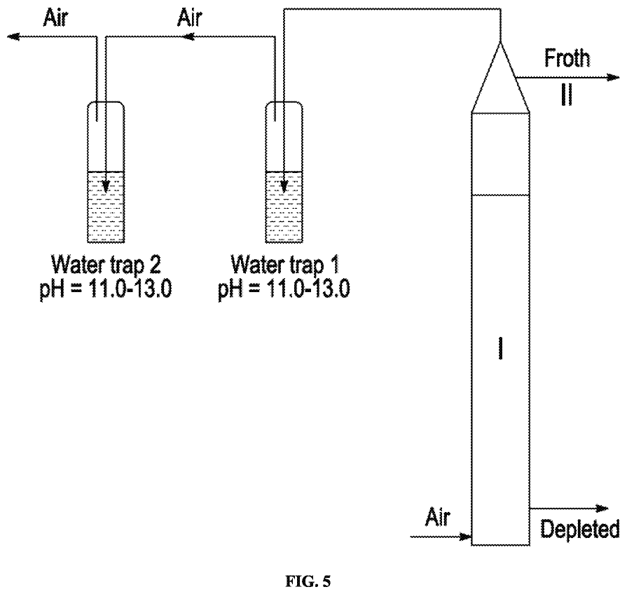

The aqueous solution can be from any suitable water source. For example, the aqueous solution can include water from a natural source (e.g., a pond, lake, river, stream, or combination thereof), an extract from contaminated soil, an extract from contaminated landfill materials, water contaminated with residual fire-fighting foam, industrial waste water (e.g., residues from the manufacture of water-repellant coatings or the application of those material to fabric or other surfaces, residues from the manufacture of non-stick coatings and from the application thereof, residue from semiconductor manufacture, residue from pesticide manufacture, residue from paint manufacture, or a residue from photography), a concentrate of any one or any combination thereof, or a combination thereof. The aqueous solution can be taken directly from a water source contaminated with the fluoroalkyl compound. In other aspects, the aqueous solution is formed by concentrating the fluoroalkyl compound from feed water, wherein the feed water can be taken directly from a water source contaminated with the fluoroalkyl compound or the feed water can be a concentrated extract of the fluoroalkyl compound derived from the water source. The aqueous solution can be a concentrate formed via the foam concentration process for concentrating PFAS described herein, various aspects of which are demonstrated in Examples 1-2 and illustrated in FIGS. 5-8. The foam concentration process can be used to both generate the initial aqueous solution and to concentrate PFAS in the treated aqueous composition that survive the initial destruction process to subject to the destruction process again. For example, feed water containing PFAS can be treated with a metal component, and then subjected to the foam concentration process in place of the treated aqueous composition described herein, to form the aqueous composition which is subjected to the destruction process to form the treated aqueous composition which can then be subjected to the foam concentration process to form a concentrate which is then subjected to the destruction process again.

The destruction procedure can be any suitable destruction procedure that destroys the one or more PFAS. The destruction procedure can include thermal treatment, treatment with an electrolytic cell, treatment with a plasma reactor, treatment with supercritical water, combustion treatment, oxidation treatment, chemical treatment, or a combination thereof. In various aspects, the destruction procedure can include electrolytically treating the aqueous composition in an electrochemical cell.

During the destruction procedure, such as during electrolytic treatment of the aqueous composition, the aqueous composition can have any suitable pH. In various aspects, the aqueous composition can have a pH of 0 to 5.5, or a pH of 0 to 5.5, or 0 to 5.2, or 1 to 4.5, or less than or equal to 5.5 and greater than or equal to 0 and less than, equal to, or greater than 0.5, 1, 1.2, 1.4, 1.5, 1.6, 1.8, 2, 2.2, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4. The treated aqueous composition can have substantially the same pH as the aqueous composition. The treated aqueous composition can have a pH of 0 to 5.5, or 0 to 5.2, or 1 to 4.5, or less than or equal to 5.5 and greater than or equal to 0 and less than, equal to, or greater than 0.5, 1, 1.2, 1.4, 1.5, 1.6, 1.8, 2, 2.2, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4.

The one or more PFAS can include any one or more PFAS. The one or more PFAS can include a perfluoroalkyl substance, a polyfluoroalkyl substance, a perfluoroalkyl acid (PFAA), or a combination thereof. The one or more PFAS can include perfluorooctanesulfonic acid (PFOA), perfluorooctyl sulfonate (PFOS), perfluorohexanesulfonic acid (PFHxS), perfluorononanoic acid (PFNA), perfluorobutanesulfonic acid (PFBS), 2-(N-methyl-perfluorooctane sulfonamido) acetic acid, perfluoroheptanoic acid (PFHpA), n-perfluorooctane sulfonic acid, perfluoromethylheptane sulfonic acid, n-perfluorooctanoic acid, a branched perfluorooctanoic acid, perfluorodecanoic acid (PFDA), perfluoroundecanoic acid (PFUn(d)A), perfluorododecanoic acid, 6:2 fluorotelomer sulfonic acid (6:2 FTS), 2,2,3,3-tetrafluoro-3-(trifluoromethoxy) propanoic acid (PFMPA), perfluoropentanesulfonic acid (PFPeS), perfluoro-4-methoxybutanoic acid (PFMBA), perfluoropentanoic acid (PFPeA), perfluorohexanoic acid (PFHxA), 4:2 fluorotelomer sulfonic acid (4:2 FTSA), perfluoroheptanesulfonic acid (PFHpS), perfluorooctanesulfonamide (PFOSA), or a combination thereof. As used herein, the term “one of more PFAS” need not refer to every PFAS that is present in the aqueous composition; for example, the term “one or more PFAS” can refer to all the PFAS that are present in the aqueous composition, or the term “one or more PFAS” can refer to a subset of the PFAS that are present in the aqueous composition.

The aqueous composition can have any suitable concentration of the one or more PFAS, such as a concentration of 14 parts per trillion (ppt) or higher, or 4 ppt or higher, or 1 part per trillion (ppt) to 100,000 parts per million (ppm), 20 ppt to 1 ppm, or less than or equal to 100,000 ppm and greater than or equal to 1 ppt and less than, equal to, or greater than 2 ppt, 3, 4, 5, 6, 7, 8, 9, 10, 11, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750, 1 ppm, 2, 4, 6, 8, 10, 15, 20, 25, 30, 35, 40, 45, 50, 60, 70, 80, 90, 100, 150, 200, 250, 300, 400, 500, 600, 700, 800, 900, 1,000, 1,500, 2,000, 5,000, 10,000, 25,000, 50,000, 75,000, or 90,000 ppm.

The treated aqueous composition can have any suitable concentration of the one or more PFAS, such as 0.000001 ppt to 90,000 ppm, or 0.01 ppt to 30,000 ppm, or less than or equal to 90,000 ppm and greater than or equal to 0.000001 ppt and less than, equal to, or greater than 0.000005 ppt, 0.00001, 0.00005, 0.0001, 0.0005, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750 ppt, 1 ppm, 2, 4, 6, 8, 10, 15, 20, 25, 30, 35, 40, 45, 50, 60, 70, 80, 90, 100, 150, 200, 250, 300, 400, 500, 600, 700, 800, 900, 1,000, 1,500, 2,000, 5,000, 10,000, 25,000, 50,000, 75,000, or 85,000 ppm.

In various aspects, the method can include performing the destruction procedure on the aqueous composition until the concentration of one or more of the one or more PFAS in the aqueous composition decreases by a certain amount, and then flowing the aqueous solution out of the destruction procedure as the treated aqueous composition, such as decreases by an amount of 1% to 100% of the original concentration (i.e., the concentration at the time the aqueous solution enters the destruction procedure), or 25% to 75%, or less than or equal to 100% and greater than or equal to 1% and less than, equal to, or greater than 2%, 3, 4, 5, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 95, 96, 97, 98, or 99%. In various aspects, the method can include performing the destruction procedure on the aqueous composition until the concentration of one or more of the one or more PFAS in the aqueous composition reaches a certain concentration, and then flowing the aqueous solution out of the destruction procedure as the treated aqueous composition, such as a concentration of 0.000001 ppt to 90,000 ppm, or 0.01 ppt to 30,000 ppm, or less than or equal to 90,000 ppm and greater than or equal to 0.000001 ppt and less than, equal to, or greater than 0.000005 ppt, 0.00001, 0.00005, 0.0001, 0.0005, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750 ppt, 1 ppm, 2, 4, 6, 8, 10, 15, 20, 25, 30, 35, 40, 45, 50, 60, 70, 80, 90, 100, 150, 200, 250, 300, 400, 500, 600, 700, 800, 900, 1,000, 1,500, 2,000, 5,000, 10,000, 25,000, 50,000, 75,000, or 85,000 ppm. The method can include in-situ monitoring of the concentration of the one or more PFAS to determine the concentration of the one or more PFAS during the destruction procedure. The determination of the percentage decrease of concentration of the one or more PFAS decrease during the destruction procedure, or of the final concentration of the one or more PFAS during the destruction procedure, can be based on the rate of destruction in the destruction procedure. For example, the determination can be based on what concentration of the one or more PFAS results in a rate of destruction that is 0.001% to 50% of the initial rate of destruction at the initial concentration of the one or more PFAS, or 1% to 25%, or less than or equal to 50% and greater than or equal to 0.001% and less than, equal to, or greater than 0.005%, 0.01, 0.05, 0.1, 0.5, 1, 2, 3, 4, 5, 6, 8, 10, 12, 14, 16, 18, 20, 22, 24, 26, 28, 30, 32, 34, 36, 38, 40, 42, 44, 46, or 48%.

In various aspects, the aqueous composition and/or the treated aqueous composition further includes a metal component. The metal component includes a metal. The metal component can include an ion of the metal, a solid or dissolved compound of the metal, an elemental form of the metal, or a combination thereof. The metal can be any suitable metal. For example, the metal can include Mg, Al, Fe, Zn, Cu, Cd, Cr, Hg, Ni, V, Ce, or a combination thereof. The metal can include Al (e.g., Al³⁺). The metal can include Mg (e.g., Mg²⁺). The metal component can include Al³⁺ or a compound of aluminum such as aluminum chloride, aluminum hydroxide, or a combination thereof. The metal component can be a salt of the metal of the metal component. The salt of the metal can be any suitable salt, such as AlCl³, Al(OH)₃, AlPO₄, Al₂(SO₄)₃, or a combination thereof. The metal component can include aluminum hydroxide. In low pH aqueous solutions, such as a pH of less than about 4, aluminum hydroxide can be dissolved, forming aluminum ions and hydroxide ions, wherein the hydroxide ions can interact with hydrogen ions to form water molecules; as such, the presence of aluminum hydroxide in aqueous solutions having a pH of less than about 4 can be indicated by the presence of aluminum ions dissolved in the aqueous solution. In higher pH aqueous solutions, such as a pH of greater than or equal to 4, aluminum hydroxide can be a solid salt (Al(OH)₃). In various aspects, the one or more PFAS in the aqueous composition can be absorbed to the metal component, adsorbed to the metal component, bonded to the metal component, coordinated to the metal component, otherwise associated with the metal component, or a combination thereof. The pH range used during the aeration and foam separation can promote formation of Al(OH)₃. The metal component can be initially added to feed water to concentrate the PFAS and to form the aqueous composition, can be added to the aqueous composition, can be added to the treated aqueous composition, or a combination thereof. For example, adding the metal component to the aqueous composition, to feed water used to generate the same, or to the treated aqueous composition can include adding an aqueous concentrate to the aqueous composition, the treated aqueous composition, or feed water that includes AlCl₃, Al(OH)₃, AlPO₄, Al₂(SO₄)₃, or a combination thereof. In various aspects, the aqueous concentrate can be formed by dissolving aluminum metal in acid, such as HCl (e.g., to form an AlCl₃ solution). The aqueous concentrate can have a low pH, such as a pH of 0 to 5.5, or 0 to 4, or 0 to 3, or 0 to 2.7, or less than or equal to 5.5 and greater than or equal to 0 and less than, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.0, 4, 4.1, 4.2, 4.3, 4.4, 4.5, 4.6, 4.7, 4.8, 4.9, 5, 5.1, 5.2, 5.3, or 5.4. The addition of the aqueous concentrate to the aqueous composition, feed water used to generate the same, or treated aqueous composition can cause the resulting composition to have a pH of 0 to 5.5 (e.g., less than or equal to 5.5 and greater than or equal to 0 and less than, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.0, 4, 4.1, 4.2, 4.3, 4.4, 4.5, 4.6, 4.7, 4.8, 4.9, 5, 5.1, 5.2, 5.3, or 5.4). Adding the metal component to the treated aqueous composition, the aqueous composition, or to a feed water used to generate the same can include adding a metal component that is recycled from a previous iteration of the method and recovered from the clarified aqueous composition thereof. The method can optionally include acidifying (e.g., via addition of acid, such as HCl, HBr, HNO₃, H₂SO₄, HClO₄, HClO₃, or a combination thereof) or basifying (e.g., via addition of base, such as NaOH, KOH, or a combination thereof) the aqueous composition or treated aqueous composition such that the aqueous composition achieves a pH of 0 to 5.5 (e.g., less than or equal to 5.5 and greater than or equal to 0 and less than, equal to, or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.0, 4, 4.1, 4.2, 4.3, 4.4, 4.5, 4.6, 4.7, 4.8, 4.9, 5, 5.1, 5.2, 5.3, or 5.4). The metal and/or metal component can have any suitable concentration in the aqueous composition or treated aqueous composition. For example, the aqueous composition or treated aqueous composition can have a concentration of the metal and/or metal component of 0.01 ppm to 800 ppm, or 0.01 ppm to 600 ppm, or 1 ppm to 500 ppm, or less than or equal to 800 ppm and greater than or equal to 0.01 ppm and less than, equal to, or greater than 0.05 ppm, 0.1, 0.2, 0.4, 0.6, 0.8, 1, 2, 3, 4, 5, 6, 8, 10, 12, 14, 16, 18, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 320, 340, 360, 380, 400, 420, 440, 460, 480, 500, 520, 540, 560, 580, 600, 650, 700, or 750 ppm.

In various aspects that include the metal component in the aqueous composition, the metal component can survive the destruction procedure and can be present in the clarified aqueous composition. The method can further include reusing the metal component in the clarified aqueous composition and/or a metal from the metal component in the clarified aqueous composition in the metal component in a subsequent iteration of the method. The reusing of the metal component and/or the metal therefrom can include flowing a recycle stream including at least part of the clarified aqueous composition or an extract thereof to or upstream of the treated aqueous composition of the subsequent iteration of the method. The reusing of the metal component and/or the metal therefrom can further include purging one or more acidification contaminants from the metal component and/or the metal therefrom prior to the reuse of the metal component and/or metal therefrom in the subsequent iteration of the method. The purging can include purging the one or more acidification contaminants from a recycle stream including at least part of the clarified aqueous composition or an extract thereof prior to the reuse of the metal component and/or metal therefrom in the subsequent iteration of the method. The purging can include separating sulfate, chloride, or a combination thereof, from the metal component and/or metal therefrom that is in the recycle stream.

At the low pH, the metal component can be substantially dissolved. In aspects wherein the aqueous composition includes the metal component, the method can further include raising a pH of the clarified aqueous composition to bring the metal component out of solution and precipitate the metal component, thereby forming a pH-adjusted aqueous composition. The pH can be adjusted by adding one or more bases to the clarified aqueous composition. The pH of the pH-adjusted aqueous composition can be raised to a pH of 3 to 12, or 6 to 6.5, or less than or equal to 12 and greater than or equal to 3 and less than, equal to, or greater than 3.5, 4, 4.5, 5, 5.5, 6, 6.5, 7, 7.5, 8, 8.5, 9, 9.5, 10, 10.5, 11, or 11.5. The method can further include separating the precipitated metal component from the pH-adjusted aqueous composition, to form a separated aqueous composition. The separated aqueous composition can be substantially free of the metal component. The precipitated metal component can be separated from the pH-adjusted aqueous composition in any suitable way, such as via filtration, centrifugation, or decantation. In aspects of the method including a recycle stream that includes the metal component or a metal therefrom therein, the method can include adding a base to a recycle stream to raise a pH thereof to 3 to 12, filtering out a precipitated salt including the one or more acidification contaminants from the recycle stream, and adding the filtered recycle stream to or upstream of the treated aqueous composition. Herein, adding upstream of the treated aqueous composition can include adding to any suitable location upstream of the treated aqueous composition, such as adding to the aqueous composition that is fed to the destruction procedure, or adding to an aqueous feed water that is treated (e.g., treated with the metal component or a metal therefrom) to remove PFAS therefrom to generate the aqueous composition that is fed to the destruction procedure. The method can include adding a base to a recycle stream including at least part of the clarified aqueous composition or an extract thereof to raise a pH thereof to 3 to 12, and filtering out a precipitated salt including the metal component from the recycle stream, and adding the precipitated salt to or upstream of the treated aqueous composition. The method can include adding an additive to the aqueous composition, such as prior to entry of the aqueous composition into the destruction process and/or while the aqueous composition is in the destruction process. The additive can include any suitable additive, such as a radical precursor, hydrogen peroxide, a sulfate salt, sulfite salt, sodium hypochlorite, potassium hypochlorite, NaCl, CaCl₂), KCl, or a combination thereof. The radical precursor can include HOOH, O₃, S₂O₈⁻, I⁻, CO₃²⁻, HCO₃⁻, H₂PO₄⁻, HPO₄²⁻, PO₄³⁻, HSO₅⁻, or a combination thereof. The additive can include a sulfate salt, a sulfite salt, a persulfate salt, or a combination thereof. The additive can include sodium sulfate, potassium sulfate, sodium persulfate, potassium persulfate, or a combination thereof. The one or more additives can have any suitable concentration in the aqueous composition, such as about 0.001 ppm to about 999,999 ppm, or about 50,000 ppm to about 140,000 ppm, or less than or equal to about 999,999 ppm and greater than or equal to about 0.001 ppm and less than, equal to, or greater than 0.001 ppm, 0.005, 0.01, 0.05, 0.1, 0.5, 1, 1.5, 2, 5, 10, 15, 20, 50, 100, 150, 200, 500, 1,000, 1,100, 1,200, 1,500, 2,000, 2,500, 5,000, 10,000, 15,000, 20,000, 50,000, 100,000, 150,000, 200,000, 500,000, 750,000, or 900,000 ppm. In various aspects, the method is free of adding the one or more additives to the aqueous composition. In various aspects, the aqueous composition is substantially free of the one or more additives.

The foaming of the treated aqueous composition to concentrate the fluoroalkyl compound can be characterized as foam fractionation, froth flotation, ion flotation, adsorptive bubble separation, bubble fractionation, multiphase separation, or a combination thereof. The fluoroalkyl compound can be an amphipathic molecule having both a hydrophilic region and a hydrophobic region. In various aspects, the fluoroalkyl compound can associate with a metal component such as aluminum hydroxide to form a hydrophobic particle that preferentially partitions to a gas-liquid interface. Under acidic conditions, complexes of the fluoroalkyl compound with ionic aluminum can form molecules having increased amphipathicity relative to the fluoroalkyl compound alone. The method can include ion flotation, wherein metal ions such as aluminum ions are captured and recovered in the foam phase along with the fluoroalkyl compound.

The method can include manipulating a surface tension or a surface energy of the treated aqueous composition to promote partitioning of the fluoroalkyl compound to the gas-liquid interface. In various aspects, reagents can be added to the aqueous solution to modify the gas-liquid interface or particle surface properties. In aspects of the method including the metal component in the treated aqueous composition, the metal component can function as a frother, a collector, an activator, a depressant, or a combination thereof, depending on the solution conditions. A frother can include a surface-active molecule that adsorbs at the gas-liquid interface to reduce surface energy and stabilize foam. A collector can include a substance that adsorbs to a surface to enhance hydrophobicity and promote attachment to gas bubbles. An activator can include a substance that promotes selective adsorption of a target species to a bubble surface. A depressant can include a substance that inhibits adsorption of a non-target species to a bubble surface.

The aerating of the treated aqueous composition can include bubbling one or more gases through the treated aqueous composition. The gas bubbled into the treated aqueous composition can be any suitable gas that forms the foam. The gas can include air, nitrogen, oxygen, ozone, argon, hydrogen, helium, or a combination thereof. In various aspects, the gas can include air. The gas can be a compressed or pressurized gas. The bubbling can include flowing the gas into an aeration apparatus that is submerged in the treated aqueous composition. The aeration apparatus can include a gas sparger, a perforated pipe, a sparging rod, a serrated cone, a diffuser ring, a stone diffuser, or a combination thereof. The aeration apparatus can include a stone diffuser, such as a porous stone that produces small bubbles when a gas is pumped therethrough. The aeration apparatus or bubble generating device can include a porous sparger, a sintered glass sparger, a sintered metal sparger, a metal frit, a perforated plate sparger, a perforated pipe, a single-orifice sparger, a multi-orifice sparger, a dynamic sparger with liquid cross-flow, a venturi, a needle wheel pump, a membrane diffuser, an ejector, a plunging liquid jet, or a combination thereof. The sparger can have a pore size of 10 microns to 1000 microns, or 10 microns to 200 microns, or less than or equal to 1000 microns and greater than or equal to 10 microns and less than, equal to, or greater than 16 microns, 20, 25, 40, 50, 60, 70, 80, 90, 100, 135, 150, 200, 250, 500, or 750 microns. Dynamic spargers utilizing liquid cross-flow can produce smaller bubble sizes than static spargers. The selection of the bubble generating device can be based on a desired bubble size distribution, a gas flow rate, a pressure drop, power consumption, or a combination thereof. The bubbling of the gas into the treated aqueous composition can form nanobubbles, microbubbles, or a combination thereof, wherein the bubbles include the gas. The bubbles can have a diameter (e.g., a diameter of one or more individual bubbles, or an number average diameter of the bubbles) of 1 nm to 10 mm, or 1 nm to 1000 nm, or 1000 nm to 1 mm, or 1 mm to 10 mm, or less than or equal to 10 mm and greater than or equal to 1 nm and less than, equal to, or greater than 2 nm, 5, 10, 20, 40, 60, 80, 100, 150, 200, 250, 300, 400, 500, 600, 700, 800, 900 nm, 1 micron, 2, 5, 10, 20, 40, 60, 80, 100, 150, 200, 250, 300, 400, 500, 600, 700, 800, 900 microns, 1 mm, 2, 3, 4, 5, 6, 7, 8, or 9 mm.

The method can include performing the bubbling of the gas into the treated aqueous composition in any suitable apparatus. In various aspects, the apparatus can include a column or tank. The apparatus can include a multi-stage column. The apparatus can include a multi-stage column with bubble cap trays. The apparatus in which the gas is bubbled into the treated aqueous composition to form the foam can include a column having a cylindrical geometry, a tapered geometry, a conical geometry, or a combination thereof. The apparatus can include a tapered section that narrows in a direction of foam rise to increase foam velocity and promote drainage. The apparatus can include multiple foam risers extending from a liquid pool zone to a foam collection zone. The apparatus can include mechanical means for enhancing foam stability or promoting foam collapse, such as rotating paddles, impellers, vibrating elements, ultrasonic transducers, spray nozzles, or humidity manipulation. The foam can be collected in any suitable way, such as using an inverted-U overflow pipe, a launder, a donut launder, a ring launder, an annular trough, a narrowed pipe overflow, a side port, a foam collection chamber, a weir, a skimmer, a vacuum extraction device, an overflow lip, or a combination thereof. The foam collection method can include combinations of flow contraction, expansion, and bending. Drainage enhancement can be achieved using internal components such as parallel inclined channels or foam riser plates that provide routes for interstitial liquid to drain back to the liquid pool.

The bubbling can be performed at a superficial gas velocity of 1 mm/s to 30 mm/s, or 1 mm/s to 12 mm/s, or 1 mm/s to 2 mm/s, or less than or equal to 30 mm/s and greater than or equal to 1 mm/s, and less than, equal to, or greater than 2 mm/s, 3, 4, 5, 6, 7, 8, 9, 10, 11, 12, 15, 20, or 25 mm/s. The gas flow rate can be 0.1 L/min to 1000 L/min, or 1 L/min to 100 L/min, or less than or equal to 1000 L/min and greater than or equal to 0.1 L/min, and less than, equal to, or greater than 0.5 L/min, 1, 2, 5, 10, 20, 50, 100, 200, 500, or 750 L/min. The gas flow rate can be selected based on a cross-sectional area of the apparatus, a volume of the treated aqueous composition, a concentration of the fluoroalkyl compound, a required throughput, or a combination thereof. The bubbles can have a Sauter mean radius of 0.1 mm to 5.5 mm, or 0.18 mm to 1 mm, or 0.5 mm to 1 mm, or less than or equal to 5.5 mm and greater than or equal to 0.1 mm, and less than, equal to, or greater than 0.15 mm, 0.18, 0.2, 0.3, 0.4, 0.5, 0.6, 0.7, 0.8, 0.9, 1.0, 1.5, 2.0, 2.5, 3.0, 4.0, or 5.0 mm. Gas residence times in the bubbly liquid pool can be 1 second to 60 seconds, or less than or equal to 60 seconds and greater than or equal to 1 second, and less than, equal to, or greater than 2 seconds, 5, 10, 15, 20, 25, 30, 40, or 50 seconds.

The gas in the headspace of the apparatus used to bubble the gas into the treated aqueous composition (which can include the portions of the gas that is bubbled into the aqueous solution but that does not form a foam and is not captured in bubbles) can be allowed to vent to the atmosphere or ambient space, or the gas in the headspace of the apparatus can be captured. In various aspects, the captured headspace gas can be treated prior to releasing to the atmosphere or ambient space, such as via treatment with basic water to capture any volatilized PFAS compounds in the gas. For example, the method can include bubbling the headspace gas through basic water prior to releasing to the atmosphere or ambient space, such as in one or more water traps (e.g., one trap, two traps, three traps, or more). The basic water can have any suitable pH, such as a pH of 8-15, or 10-14, or less than or equal to 15 and greater than or equal to 8 and less than, equal to, or greater than 8.5, 9, 9.5, 10, 10.5, 11, 11.5, 12, 12.5, 13, 13.5, 14, or 14.5.

The gas in the generated foam can include volatilized PFAS compounds. After breaking the foam or allowing the bubbles of the foam to dissipate, the released gas from the broken or dissipated foam can be allowed to vent to the atmosphere or ambient space, or the released gas can be captured. In various aspects, the captured released gas can be treated prior to releasing to the atmosphere or ambient space, such as via treatment with basic water to capture any volatilized PFAS compounds in the gas. For example, the method can include bubbling the released gas through basic water prior to releasing to the atmosphere or ambient space, such as in one or more water traps (e.g., one trap, two traps, three traps, or more). The basic water can have any suitable pH, such as a pH of 8-15, or 10-14, or less than or equal to 15 and greater than or equal to 8 and less than, equal to, or greater than 8.5, 9, 9.5, 10, 10.5, 11, 11.5, 12, 12.5, 13, 13.5, 14, or 14.5.

The foam removed from the treated aqueous composition forms the concentrate that includes the foam. The concentrate is an aqueous concentrate. The removing of the foam from the treated aqueous composition to form the aqueous concentrate can be conducted in any suitable manner. The removing of the foam from the treated aqueous composition can include scooping the foam from a surface of the treated aqueous composition, vacuuming the foam from a surface of the treated aqueous composition, pushing the foam off a surface of the treated aqueous composition, or a combination thereof. The method can further include breaking the foam (e.g., substantially eliminating the gas bubbles therein), to form the aqueous concentrate. The aqueous concentrate can have a concentration of the one or more PFAS from the treated aqueous composition that is greater than the concentration of the one or more PFAS in the treated aqueous composition. The aqueous concentrate can have a concentration of the one or more PFAS from the treated aqueous composition, of 0.005 parts-per-trillion (ppt) to 500,000 parts-per-million (ppm), or 0.005 ppt to 1,000 ppm, or less than or equal to 500,000 ppm and greater than or equal to 0.005 ppt and less than, equal to, or greater than 0.01 ppt, 0.05, 0.1, 0.5, 1, 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 400, 500, 750 ppt, 1 ppb, 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 400, 500, 750 ppb, 1 ppm, 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 400, 500, 750, 1,000, 1,500, 2,000, 5,000, 10,000, 15,000, 20,000, 40,000, 60,000, 80,000, 100,000, 150,000, 200,000, 300,000, or 400,000 ppm. Herein, concentration of the one or more PFAS in the concentrate can be measured with the concentrate substantially free of bubbles and foam. A ratio of a concentration of the one or more PFAS in the treated aqueous composition to a concentration of the one or more PFAS in the concentrate can be 1:2 to 1:2,000,000, or 1:2 to 1:100,000, or 1:5 to 1:40, or less than or equal to 1:2 and greater than or equal to 1:2,000,000 and less than, equal to, or greater than 1:1,000,000, 1:900,000, 1:800,000, 1:700,000, 1:600,000, 1:500,000, 1:400,000, 1:300,000, 1:200,000, 1:100,000, 1:80,000, 1:60,000, 1:40,000, 1:20,000, 1:10,000, 1:5,000, 1:1,000, 1:900, 1:800, 1:700, 1:600, 1:500, 1:400, 1:300, 1:200, 1:100, 1:90, 1:80, 1:70, 1:65, 1:60, 1:55, 1:50, 1:48, 1:46, 1:45, 1:44, 1:42, 1:30, 1:38, 1:36, 1:35, 1:34, 1:32, 1:30, 1:28, 1:26, 1:25, 1:24, 1:22, 1:20, 1:18, 1:16, 1:15, 1:14, 1:12, 1:10, 1:9, 1:8, 1:7, 1:6, 1:5, 1:4, or 1:3. A ratio of the volume of the treated aqueous composition with respect to the volume of the aqueous concentrate produced therefrom can substantially correspond to the ratio of the concentration of the one or more PFAS in the treated aqueous composition to the concentration of the one or more PFAS in the aqueous concentrate. For example, the ratio of the volume of the treated aqueous composition with respect to the volume of the aqueous concentrate can be 1:2 to 1:2,000,000, or 1:2 to 1:100,000, or 1:5 to 1:40, or less than or equal to 1:2 and greater than or equal to 1:2,000,000 and less than, equal to, or greater than 1:1,000,000, 1:900,000, 1:800,000, 1:700,000, 1:600,000, 1:500,000, 1:400,000, 1:300,000, 1:200,000, 1:100,000, 1:80,000, 1:60,000, 1:40,000, 1:20,000, 1:10,000, 1:5,000, 1:1,000, 1:900, 1:800, 1:700, 1:600, 1:500, 1:400, 1:300, 1:200, 1:100, 1:90, 1:80, 1:70, 1:65, 1:60, 1:55, 1:50, 1:48, 1:46, 1:45, 1:44, 1:42, 1:30, 1:38, 1:36, 1:35, 1:34, 1:32, 1:30, 1:28, 1:26, 1:25, 1:24, 1:22, 1:20, 1:18, 1:16, 1:15, 1:14, 1:12, 1:10, 1:9, 1:8, 1:7, 1:6, 1:5, 1:4, or 1:3.

The clarified aqueous composition is the treated aqueous composition having the foam removed therefrom. The clarified aqueous composition has a concentration of the one or more PFAS from the treated aqueous composition that is lower than the concentration of the one or more PFAS in the treated aqueous composition. The clarified aqueous composition can have a concentration of the one or more PFAS of 0.0001 parts-per-trillion (ppt) to 100 parts-per-million (ppm), or 0.0001 ppt to 10 ppt, or 0.0001 ppt to 4 ppt, or less than or equal to 100 ppm and greater than or equal to 0.0001 ppt or less than, equal to, or greater than 0.0002 ppt, 0.0004, 0.0006, 0.0008, 0.001, 0.005, 0.01, 0.05, 0.1, 0.2, 0.4, 0.5, 0.6, 0.8, 1, 1.5, 2, 2.5, 3, 3.5, 4, 4.5, 5, 6, 7, 8, 9, 10, 11, 12, 13, 14, 15, 16, 17, 18, 19, 20, 25, 30, 40, 50, 60, 70, 80, 90, 100, 150, 200, 250, 300, 400, 500, 750 ppt, 1 ppb, 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 400, 500, 750 ppb, 1 ppm, 2, 3, 4, 5, 6, 7, 8, 9, 10, 11, 12, 13, 14, 15, 20, 25, 30, 35, 40, 45, 50, 60, 70, 80, or 90 ppm. A ratio of a concentration of the one or more PFAS in the clarified aqueous composition to a concentration of the one or more PFAS in the treated aqueous composition can be 1:2 to 1:1000, or 1:5 to 1:40, or less than or equal to 1:2 and greater than or equal to 1:1,000 and less than, equal to, or greater than 1:900, 1:800, 1:700, 1:600, 1:500, 1:400, 1:300, 1:200, 1:100, 1:90, 1:80, 1:70, 1:65, 1:60, 1:55, 1:50, 1:48, 1:46, 1:45, 1:44, 1:42, 1:30, 1:38, 1:36, 1:35, 1:34, 1:32, 1:30, 1:28, 1:26, 1:25, 1:24, 1:22, 1:20, 1:18, 1:16, 1:15, 1:14, 1:12, 1:10, 1:9, 1:8, 1:7, 1:6, 1:5, 1:4, or 1:3.

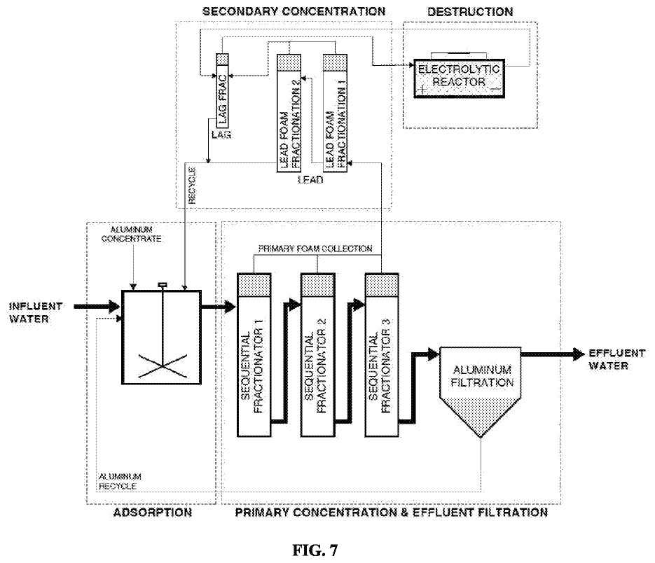

The method can be performed in a batch mode, a semi-batch mode, a continuous mode, a stripping mode, or a combination thereof. In batch mode, a fixed volume of the treated aqueous composition can be subjected to the bubbling, foam can be generated to a desired height, gas supply can be stopped, and the foam can be allowed to drain before collection. In semi-batch mode, the treated aqueous composition can be charged batchwise to the apparatus while gas is sparged continuously, and foam can be collected continuously or periodically at the top of a column while the liquid pool becomes depleted over time. A secondary concentration step (e.g., bubbling gas into and removing foam from a concentrate formed from a prior iteration of bubbling and foam removal, as shown in FIG. 7) can be performed in semi-batch mode. In continuous mode, the treated aqueous composition can be fed continuously to the apparatus and the clarified aqueous composition is withdrawn continuously while foam is collected. A primary concentration step (e.g., bubbling gas into the treated aqueous composition and removing foam therefrom, as shown in FIG. 7) can be performed in continuous mode or a variation thereof. Continuous mode can include external reflux, wherein a portion of collected foamate can be returned to the top of a column as washwater. In stripping mode, a feed stream can be added to the top of a foam column and stripped by rising gas. The method can include internal reflux, wherein liquid can be liberated within the column by bubble coalescence or disproportionation and flows countercurrent to the rising foam. The method can include bubble fractionation, wherein species can be concentrated toward the top of a bubbly liquid layer by adsorption to the surface of rising bubbles without the formation of a stable foam layer, and the enriched liquid near the surface can be removed. Although FIG. 7 illustrates an aspect of the present disclosure wherein influent water (e.g., feed water) is treated with aluminum concentrate and the resulting product is fed to sequential fractionator 1, aspects of the present disclosure also encompass a modified FIG. 7 wherein the treatment of influent water with aluminum concentrate is replaced by the destruction process described herein (e.g., any destruction process and not limited to electrolytic destruction), the treated aqueous composition formed by the destruction process is fed to sequential fractionator 1, and the destruction process that follows the secondary concentration shown in FIG. 7 is the same destruction process used to form the treated aqueous composition fed to sequential fractionator 1.

The method can include controlling a position of an interface between a bubbly liquid zone and a foam zone in the apparatus in which the bubbling is performed. The controlling can include using one or more sensors including a pressure transducer, a level transmitter, a conductivity sensor, an optical sensor, an ultraviolet (UV) absorption sensor, or a combination thereof. The controlling can include using a feedback control loop such as a proportional-integral-derivative (PID) control loop to modulate one or more of a gas flow rate, a valve position, an outlet weir height, a liquid feed rate, a liquid withdrawal rate, or a combination thereof. The controlling can include using automated flow control valves, variable-speed pumps, or a combination thereof. Inventory control can be managed through a vented underflow arrangement, wherein the pressure of a liquid pool in the apparatus is maintained by raising the outlet level of a bottom rejectate stream using a vented T-piece, allowing for adjustment of the gauge pressure at the bottom of the column.

The method can include placing the concentrate back into the destruction process for further destruction of PFAS. The concentrate can be placed back into the destruction process alone. The concentrate can be placed back into the destruction process combined with other concentrates from other performances of the destruction process on other batches or portions of the aqueous composition. The concentrate can be placed back into the destruction process combined with other batches or portions of the aqueous composition. In various aspects, placing the concentrate into the destruction process can form a second treated aqueous composition that has a concentration of the one or more PFAS that is 0.0001% to 90% of the concentration of the one or more PFAS in the concentrate, or 1% to 30%, or less than or equal to 90% and greater than or equal to 0.0001% and less than, equal to, or greater than 0.0005%, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 65, 70, 75, 80, or 85% of the concentration of the one or more PFAS in the concentrate. The method can include repeating the destruction process, aeration, separation of foam, and placement of the concentrate back into the destruction process for further destruction of PFAS 2 times, 3 times, 4, 5, 6, 7, 8, 9, or 10 or more times.

The method can include performing the destruction process on the aqueous composition until the concentration of the one or more PFAS in the aqueous composition reaches a predetermined concentration (e.g., a predetermined proportion of an original concentration). The predetermined concentration can be any suitable concentration, such as 0.0001% to 90% of a starting concentration of PFAS in the aqueous composition, or 1% to 30%, or less than or equal to 90% and greater than or equal to 0.0001% and less than, equal to, or greater than 0.0005, 0.001, 0.005, 0.01, 0.05, 0.1, 0.5, 1, 2, 3, 4, 5, 6, 8, 10, 12, 14, 16, 18, 20, 25, 30, 35, 40, 45, 50, 55, 60, 65, 70, 75, 80, or 85% of a starting concentration of PFAS in the aqueous composition. Whether the aqueous composition has reached a predetermined concentration, such as a predetermined proportion of an original concentration, can be measured via any suitable monitoring/measuring technique.

The method can include foaming the treated aqueous composition, collecting the first foam, then foaming the residual aqueous composition again and collecting the second foam. The first and second foams can be combined to form the concentrate. The residual aqueous composition having the second foam removed therefrom can be the clarified aqueous composition. The foaming of the “bottoms” can be repeated any suitable number of times to achieve a desired concentration of the one or more PFAS in the clarified aqueous composition (e.g., repeated equal to, less than, or greater than 1 time, 2 times, 3, 4, 5, 6, 7, 8, 9, or 10 times). The repeated foaming of the “bottoms” is illustrated in FIG. 7 as the primary concentration step performed by sequential fractionators 1-3, wherein sequential fractionator 1 performs the first iteration, and sequential fractionators 2-3 perform the second and third iteration, respectively. For example, in various aspects, the treated aqueous composition having had the foam removed therefrom can be a second aqueous composition, and the method can further include bubbling the gas into the second aqueous composition to form a second foam and removing the second foam from the second aqueous composition, and combining the foam removed from the treated aqueous composition and the second foam to form the concentrate including the one or more PFAS, and the second aqueous composition having the second foam removed therefrom can be the clarified aqueous composition. In various aspects, the second aqueous composition having had the second foam removed therefrom can be a third aqueous composition, and the method can further include bubbling the gas into the third aqueous composition to form a third foam and removing the third foam from the third aqueous composition, and combining the foam removed from the treated aqueous composition, the second foam, and the third foam to form the concentrate including the one or more PFAS and wherein the third aqueous composition having the foam removed therefrom is the clarified aqueous composition. In various aspects, the method can include repeating sequential foaming of the treated aqueous composition more than two times, and combining the removed foams to form the concentrate including the one or more PFAS, and wherein the sequentially foamed treated aqueous composition having the foams removed therefrom is the clarified aqueous composition.

The method can include performing the method once and not more than one time. In other aspects, the method can include performing the method two or more times (e.g., a first time, and a subsequent iteration), using the aqueous concentrate as the treated aqueous composition in each iteration of the two or more times. In an aspect including performing the method two or more times, the method can include performing the bubbling of the gas into the aqueous concentrate to form the foam and the removal of the foam to form the concentrate and the clarified aqueous composition using the concentrate from a previous iteration (e.g., first, or subsequent) of the foaming. In other words, the method can include performing the bubbling of gas into a concentrate from a previous iteration of the foaming to form the foam and removing the foam therefrom, such that the removed foam is the concentrate and the liquid having foam removed therefrom is the clarified aqueous composition. For example, the method can include performing a first iteration including bubbling a gas into an aqueous composition including the one or more PFAS to form a foam including the one or more PFAS, and removing the foam from the aqueous composition to form a concentrate including the one or more PFAS and to form a clarified aqueous composition. The method can then include performing a second iteration including bubbling the gas into the concentrate including the one or more PFAS to form a secondary foam including the one or more PFAS and removing the secondary foam from the concentrate to form a secondary concentrate including the one or more PFAS and to form a secondary clarified aqueous composition. Performing the bubbling and removing multiple times using the formed concentrate as the treated aqueous composition corresponds to the secondary concentration step shown in FIG. 7, wherein the sequential fractionators 1-3 in the primary concentration represent the first iteration, the lead foam fractionators 1-2 in the secondary concentration represent the second iteration, and the lag fractionator in the secondary concentration represent the third iteration. The method can include recycling the clarified aqueous composition formed from the second or higher iteration of the two or more times back to the treated aqueous composition used in the first foaming step of the method; such recycling corresponds to the recycle stream in FIG. 7 from the lead foam fractionator 2 in the secondary concentration step. During a second or higher iteration of the two or more times, a pH of the treated aqueous composition can be 0 to 5.5, or 0 to 5.2, or 2.5 to 4.0, or less than or equal to 5.5 and greater than or equal to 0 and less than, equal to, or greater than 0.5, 1, 1.2, 1.4, 1.5, 1.6, 1.8, 2, 2.2, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4. For example, in FIG. 7, the pH of the aqueous composition in the secondary concentration step can be 0 to 5.5, or 2.5 to 4.0. A ratio of a concentration of the one or more PFAS in the treated aqueous composition used in the first iteration of the two or more times to a concentration of the one or more PFAS in the concentrate formed by the last iteration of the two or more times can be 1:4 to 1:100,000, or 1:10 to 1:80, or less than or equal to 1:4 and greater than or equal to 1:100,000 or less than, equal to, or greater than 1:80,000, 1:60,000, 1:40,000, 1:20,000, 1:10,000, 1:5,000, 1:1,000, 1:900, 1:800, 1:700, 1:600, 1:500, 1:400, 1:300, 1:200, 1:100, 1:90, 1:80, 1:70, 1:65, 1:60, 1:55, 1:50, 1:48, 1:46, 1:45, 1:44, 1:42, 1:30, 1:38, 1:36, 1:35, 1:34, 1:32, 1:30, 1:28, 1:26, 1:25, 1:24, 1:22, 1:20, 1:18, 1:16, 1:15, 1:14, 1:12, 1:10, 1:9, 1:8, 1:7, 1:6, or 1:5. A ratio of the volume of the treated aqueous composition used in the first iteration of the two or more times to the volume of the concentrate formed by the last iteration of the method can substantially correspond to the ratio of the concentration of the one or more PFAS in the treated aqueous composition used in the first iteration of the two or more times to the concentration of the one or more PFAS in the concentrate formed by the last iteration of the two or more times. For example, the ratio of the volume of the treated aqueous composition used in the first iteration of the two or more times to the volume of the concentrate formed by the last iteration of the method can be 1:4 to 1:100,000, or 1:10 to 1:80, or less than or equal to 1:4 and greater than or equal to 1:100,000 or less than, equal to, or greater than 1:80,000, 1:60,000, 1:40,000, 1:20,000, 1:10,000, 1:5,000, 1:1,000, 1:900, 1:800, 1:700, 1:600, 1:500, 1:400, 1:300, 1:200, 1:100, 1:90, 1:80, 1:70, 1:65, 1:60, 1:55, 1:50, 1:48, 1:46, 1:45, 1:44, 1:42, 1:30, 1:38, 1:36, 1:35, 1:34, 1:32, 1:30, 1:28, 1:26, 1:25, 1:24, 1:22, 1:20, 1:18, 1:16, 1:15, 1:14, 1:12, 1:10, 1:9, 1:8, 1:7, 1:6, or 1:5.

During performance of a second or higher iteration of the bubbling and the removing of the foam using the formed concentrate as the treated aqueous composition of the next iteration, the method can include foaming the treated aqueous composition, collecting the first foam, then foaming the residual aqueous composition again and collecting the second foam. The first and second foams can be combined to form the concentrate. The residual aqueous composition having the second foam removed therefrom can be the clarified aqueous composition. The foaming of the “bottoms” from the second or higher iteration of the bubbling and the removing of the foam using the formed concentrate as the treated aqueous composition of the next iteration can be repeated any suitable number of times to achieve a desired concentration of the one or more PFAS in the clarified aqueous composition (e.g., repeated equal to, less than, or greater than 1 time, 2 times, 3, 4, 5, 6, 7, 8, 9, or 10 times). The repeated foaming of the “bottoms” from the second or higher iteration of the bubbling and the removing of the foam using the formed concentrate as the treated aqueous composition of the next iteration is illustrated in FIG. 7 in the secondary concentration step as performed by lead foam fractionator 2, wherein sequential fractionators 1-3 perform the first iteration of the bubbling and removing of the foam, and wherein the concentrate therefrom then is bubbled and the foam removed in lead foam fractionator 1, and the “bottoms” from lead form fractionator 1 is then foamed in lead foam fractionator 2. The bubbling and removing of foam from the “bottoms” can be repeated more than once; for example, during the second or higher iteration of the two or more times, the second aqueous composition having had the second foam removed therefrom is a third aqueous composition, further including bubbling the gas into the third aqueous composition to form a third foam and removing the third foam from the third aqueous composition, and combining the foam removed from the treated aqueous composition, the second foam, and the third foam to form the concentrate including the one or more PFAS and wherein the third aqueous composition having the foam removed therefrom is the clarified aqueous composition (such an embodiment would correspond to the apparatus in FIG. 7 as a lead foam fractionator 3, which is not shown). The method can include, during the second or higher iteration of the two or more times, repeating sequential foaming of the treated aqueous composition more than two times, and combining the removed foams to form the concentrate including the one or more PFAS, and wherein the sequentially foamed treated aqueous composition having the foams removed therefrom is the clarified aqueous composition. The method can include recycling the clarified aqueous composition formed form the second or higher iteration of the two or more times back to the treated aqueous composition used in the first foaming step of the method; such recycling corresponds to the recycle stream in FIG. 7 from the lead foam fractionator 2 in the secondary concentration step.

After performance of a second or higher iteration of the bubbling and the removing of the foam using the formed concentrate as the treated aqueous composition of the next iteration, the concentrate formed by the second or higher iteration of the bubbling and the removing of the foam can be used as the treated aqueous composition in a third or higher iteration of the bubbling and the removing of the foam. Such a third iteration corresponds to the lag fractionator shown in FIG. 7, which uses the concentrate formed by lead foam fractionators 1-2 as the treated aqueous composition thereof. For example, the method can include performing the bubbling of the gas into the treated aqueous composition including the one or more PFAS to form the foam including the one or more PFAS and the removing of the foam from the treated aqueous composition to form the concentrate including the one or more PFAS and to form the clarified aqueous composition one or more additional times using the concentrate formed by combining the foam and any additional foams as the treated aqueous composition in each of the one or more additional times. The method can include recycling the clarified aqueous composition formed from performing the bubbling of the gas into the treated aqueous composition including the one or more PFAS to form the foam including the one or more PFAS and the removing of the foam from the treated aqueous composition to form the concentrate including the one or more PFAS and to form the clarified aqueous composition the one or more additional times back to the treated aqueous composition used in the first foaming step of the method; such recycling corresponds to the recycling stream in FIG. 7 shown coming from the lag fractionator.

In various aspects, the treated aqueous composition having had the foam removed therefrom is a second aqueous composition, and the method further includes bubbling the gas into the second aqueous composition to form a second foam and removing the second foam from the second aqueous composition, and combining the foam removed from the treated aqueous composition and the second foam to form the concentrate including the one or more PFAS, and wherein the second aqueous composition having the second foam removed therefrom is the clarified aqueous composition; this corresponds to sequential fractionators 1-2 in FIG. 7. The method can include performing the bubbling of the gas into the treated aqueous composition including the one or more PFAS to form the form including the one or more PFAS and the removing of the foam from the treated aqueous composition to form the concentrate including the one or more PFAS and to form a clarified aqueous composition two or more times using the concentrate as the treated aqueous composition in each iteration of the two or more times, wherein during a second or higher iteration of the two or more times a pH range of the treated aqueous solution is 0 to 5.5, or 0 to 5.2, or 2.5 to 4.0, or less than or equal to 5.5 and greater than or equal to 0 and less than, equal to, or greater than 0.5, 1, 1.2, 1.4, 1.5, 1.6, 1.8, 2, 2.2, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4; this corresponds to lead foam fractionator 1 in FIG. 7. During a second or higher iteration of the two or more times, the treated aqueous composition having had the foam removed therefrom can be a secondary second aqueous composition, and the method can further include bubbling the gas into the secondary second aqueous composition to form a secondary second foam and removing the secondary second foam from the secondary second aqueous composition, and combining the foam removed from the treated aqueous composition in the first iteration of the two or more times and the secondary second foam to form the concentrate including the one or more PFAS, and wherein the secondary second aqueous composition having the secondary second foam removed therefrom is a secondary clarified aqueous composition; this corresponds to lead foam fractionator 2 in FIG. 7. The method can include recycling the secondary clarified aqueous composition formed from the second or higher iteration of the two or more times back to the treated aqueous composition used in the first foaming step of the method; this corresponds to the recycle stream shown in FIG. 7 coming from lead foam fractionator 2.

In various aspects of the method of the present disclosure, the aqueous composition is subjected to the destruction process to form the treated aqueous composition, the aerating of the treated aqueous composition is performed, the foam is separated to form a concentrate and a clarified aqueous composition, and the concentrate is placed directly back into the destruction process without any further concentration of the one or more PFAS therein. The destruction process can be performed at any suitable pH, such as a pH of 0 to 5.5, or 1 to 3, or less than or equal to 5.5 and greater than or equal to 0 and less than, equal to, or greater than 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4. The treated aqueous composition can be aerated under pH conditions of 0 to 5.5, or 4.5 to 5.3, or 0 to 5.5, or 1 to 2, or less than or equal to 5.5 and greater than or equal to 0 and less than, equal to, or greater than 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4. The treated aqueous composition can be aerated at higher pH than the destruction step to encourage formation of a metal salt, such as aluminum hydroxide, which can aid in adsorption of the one or more PFAS during the aeration. The unit wherein the aeration is performed can correspond to the “lag frac” shown in FIG. 7. The aqueous composition that is subjected to the destruction process can be a concentrate formed by aerating a first aqueous composition (e.g., influent water treated with aluminum hydroxide) at a pH of 0 to 5.5 (e.g., 4.5 to 5.3, or less than or equal to 5.5 and greater than or equal to 0 and less than, equal to, or greater than 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4) to form a primary foam (e.g., primary concentrate) and a first clarified aqueous composition (e.g., wherein the first aqueous composition can optionally be aerated multiple times as shown by sequential fractionators 1-3 in FIG. 7 to form multiple foams that can be combined to form the primary concentrate), and then aerating the primary concentrate at a pH of 0 to 5.5 (e.g., 2.5 to 4.0, or less than or equal to 5.5 and greater than or equal to 0 and less than, equal to, or greater than 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4) to form a secondary foam (e.g., secondary concentrate, wherein the primary concentrate can optionally be aerated multiple times as shown by lead foam fractionators 1-2 to form multiple foams that can be combined to form the secondary concentrate), and then aerating the secondary concentrate to form a tertiary foam that is the aqueous composition, wherein the tertiary foam corresponds to the foam generated in the lag fractionator shown in FIG. 7, and wherein a foam that is subjected to aeration can be broken (i.e., defoamed) prior to aeration. As such, in various aspects, the same fractionation apparatus can be used to form the aqueous composition and for aeration of the treated aqueous composition. The pH of the lag fractionator can be higher (e.g., 4.5 to 5.3) during aeration of the treated aqueous composition than during aeration of the secondary concentrate (e.g., 2.5 to 4) to encourage formation of solid aluminum hydroxide during aeration of the treated aqueous composition.

The combination of treating an aqueous composition with a destruction process and aeration can form a clarified aqueous composition having a low concentration of the PFAS more efficiently than performing the destruction process alone on the aqueous composition to generate the same low concentration of PFAS. The concentrate formed by the method can then be re-subjected to the destruction process for further destruction of PFAS. By only performing the destruction process on an aqueous solution having a significant concentration of PFAS, and by avoiding performing the destruction process on an aqueous composition with dilute concentrations of PFAS wherein the energy spent on the destruction process has diminishing returns, the combination of the destruction process and aeration can more efficiently reduce PFAS concentrations as compared to using the destruction process alone. Performing the destruction process to destroy the PFAS in the aqueous composition and iteratively performing the destruction process to destroy the PFAS in the concentrate formed in the method can destroy PFAS more efficiently compared to a single step of the destruction process, including because the destruction process is performed at higher concentrations of PFAS when the destruction process is more efficient.

The method can have a destruction efficiency, comparing the concentration of the one or more PFAS in the aqueous composition to the concentration of the one or more PFAS in the clarified aqueous composition, of 50% to 100%, or 95% to 100%, or less than or equal to 100% and greater than or equal to 50% and less than, equal to, or greater than 51%, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.2, 99.4, 99.6, 99.8, 99.9, 99.95, 99.99, or 99.999%. The destruction efficiency can apply to all of the one or more PFAS in the aqueous composition, or the destruction efficiency can apply to one or a subset of the one or more PFAS in the aqueous composition.

### Electrolytic Destruction Process.

In various aspects, the destruction procedure can include electrolytically treating the aqueous composition in an electrochemical cell. The electrolytically-treated aqueous composition can have a concentration of the one or more PFAS that is 0.0001% to 90% of a concentration of the one or more PFAS in the aqueous composition, or 1% to 30%, or less than or equal to 90% and greater than or equal to 0.0001% and less than, equal to, or greater than 0.0005%, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, 50, 55, 60, 65, 70, 75, 80, or 85% of a concentration of the one or more PFAS in the aqueous composition. The electrolytically-treated aqueous composition can have any suitable concentration of the one or more PFAS, such as a concentration of 0.000001 ppt to 90,000 ppm, or 0.01 ppt to 30,000 ppm, or less than or equal to 90,000 ppm and greater than or equal to 0.000001 ppt and less than, equal to, or greater than 0.000005 ppt, 0.00001, 0.00005, 0.0001, 0.0005, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750 ppt, 1 ppm, 2, 4, 6, 8, 10, 15, 20, 25, 30, 35, 40, 45, 50, 60, 70, 80, 90, 100, 150, 200, 250, 300, 400, 500, 600, 700, 800, 900, 1,000, 1,500, 2,000, 5,000, 10,000, 25,000, 50,000, 75,000, or 85,000 ppm.

Electrolytic treatment of the aqueous composition destroys at least some of the one or more PFAS in the aqueous composition, which causes the concentration of the one or more PFAS in the electrolytically-treated aqueous composition to be lower than the concentration of the one or more PFAS in the aqueous composition. The destruction can occur in any suitable way. In various aspects, the electrolytic treatment of the aqueous composition oxidizes and/or mineralizes the one or more PFAS, thereby transforming the one or more PFAS into materials including carbon dioxide and fluoride. In various aspects, the destruction products of destroying the one or more PFAS are substantially all carbon dioxide and fluoride. The method can include removing carbon dioxide and/or fluoride from the treated aqueous composition and/or from the clarified aqueous composition.

The electrolytically-treated aqueous composition separated from the foam can have a concentration of the one or more PFAS that is 0.0001% to 50% of a concentration of the one or more PFAS in the separated foam, or 0.001% to 10%, or less than or equal to 50% and greater than or equal to 0.0001% and less than, equal to, or greater than 0.0005%, 0.001, 0.005, 0.01, 0.05, 0.1, 0.5, 1, 2, 3, 4, 5, 6, 7, 8, 9, 10, 12, 14, 16, 18, 20, 25, 30, 35, 40, or 45% of a concentration of the one or more PFAS in the separated foam. The electrolytically-treated aqueous composition separated from the foam can have any suitable concentration of the one or more PFAS, such as a concentration of the one or more PFAS 0.000001 ppt (e.g., undetectable) to 45,000 ppm, or 0.01 ppt (e.g., undetectable) to 10,000 ppm, or 0.001 ppt to 100 ppt, or 0.001 ppt to 15 ppt, or less than or equal to 45,000 ppm and greater than or equal to 0.0001% of 0.000001 ppt and less than, equal to, or greater than 0.000005 ppt, 0.00001, 0.00005, 0.0001, 0.0005, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 22, 24, 26, 28, 30, 35, 40, 45, 50, 60, 70, 80, 90, 100, 150, 200, 250, 300, 400, 500, 750 ppt, 1 ppm, 2, 4, 6, 8, 10, 15, 20, 25, 30, 35, 40, 45, 50, 60, 70, 80, 90, 100, 150, 200, 250, 300, 400, 500, 600, 700, 800, 900, 1,000, 1,500, 2,000, 5,000, 10,000, 20,000, 30,000, or 40,000 ppm.

The method can include performing the electrolytic treatment on the aqueous composition using the electrolytic cell until the concentration of the one or more PFAS in the aqueous composition reaches a predetermined concentration (e.g., a predetermined proportion of an original concentration). The predetermined concentration can be any suitable concentration, such as 0.0001% to 90% of a starting concentration of PFAS in the aqueous composition, or 1% to 30%, or less than or equal to 90% and greater than or equal to 0.0001% and less than, equal to, or greater than 0.0005, 0.001, 0.005, 0.01, 0.05, 0.1, 0.5, 1, 2, 3, 4, 5, 6, 8, 10, 12, 14, 16, 18, 20, 25, 30, 35, 40, 45, 50, 55, 60, 65, 70, 75, 80, or 85% of a starting concentration of PFAS in the aqueous composition. Whether the aqueous composition has reached a predetermined concentration, such as a predetermined proportion of an original concentration, can be measured via any suitable monitoring/measuring technique.

The aqueous composition can have any suitable pH during treatment with the electrolytic cell. The aqueous composition can have a pH of 0 to 5.5, or 1 to 3, or less than or equal to 5.5 and greater than or equal to 0 and less than, equal to, or greater than 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4.

In various aspects, the method includes using a trap including water in the headspace of the electrolytic cell, such that release of volatile or gaseous material from the aqueous composition passes through the water in the trap. The water can have any suitable pH, such as a pH of 6 to 12, or 8 to 12, or less than or equal to 12 and greater than or equal to 6 and less than, equal to, or greater than 6.5, 7, 7.5, 8, 8.5, 9, 9.5, 10, 10.5, 11, or 11.5. The monitoring can include measuring a concentration of PFAS in the water of the water-filled trap, such as via ion chromatography, and using the measured concentration of PFAS in the water of the water-filled trap to estimate the concentration of the one or more PFAS in the aqueous composition, or to estimate the change in concentration in terms of a proportion of the original concentration of the one or more PFAS in the aqueous composition.

The electrolytic cell can include an electrolytic anode and an electrolytic cathode having a gap therebetween such that they are free of direct contact with one another. The gap (e.g., the distance between major faces of the electrolytic anode and electrolytic cathode) can have any suitable size, such as a size of 0.5 mm to 10 mm, or 1 mm to 3 mm, or less than or equal to 10 mm and greater than or equal to 0.5 mm and less than, equal to, or greater than 0.6 mm, 0.8, 1, 1.2, 1.4, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.2, 4.4, 4.6, 4.8, 5, 5.5, 6, 6.5, 7, 7.5, 8, 8.5, 9, or 9.5 mm. The electrolytic anode can include an anode material including a metal oxide, a transition metal oxide, a mixed metal oxide (MMO), Ti₄O₇, PbO₂, boron-doped diamond (BDD), SnO₂, Bi₂O₃, RuO₂, IrO₂, TiO₂, Ta₂O₅, a precious metal, platinum (e.g., platinum coating on titanium), PtO₂, MnO₂, CeO₂, Rh₂O₃, carbon (e.g., BDD, graphite, graphene, or a combination thereof), or a combination thereof. For example, the electrolytic anode can include RuO₂ and IrO₂, or can include PbO₂ and Bi₂O₃, or can include IrO₂, RuO₂, PtO₂, and Rh₂O₃, or can include TiO₂, RuO₂ and IrO₂. The electrolytic anode can be formed entirely of the anode material (e.g., and is free of catalyst coatings) or the electrolytic anode can include a coating or deposition of the anode material (e.g., as a catalyst coating) on a suitable substrate such as titanium, stainless steel, carbon steel, carbon (e.g., BDD, graphite, graphene, or a combination thereof), or a combination thereof. The electrolytic anode can include titanium plate coated with titanium oxide/ruthenium-iridium oxide. The catalyst coating can be on one major face of the electrolytic anode or on both major faces. The electrolytic cathode can include stainless steel, titanium, carbon (e.g., BDD, graphite, graphene, or a combination thereof), carbon steel, a precious metal, platinum, nickel, iron, copper, silver, or a combination thereof. The electrolytic cathode can be formed entirely of the cathode material (e.g., and is free of catalyst coatings) or the electrolytic cathode can include a coating of the cathode material (e.g., as a catalyst coating) on a suitable substrate, such as on a substrate that is titanium, stainless steel, carbon steel, Pt, Ni, Fe, Cu, Ag, carbon, or a combination thereof. The catalyst coating can be on one major face of the electrolytic cathode or on both major faces. The electrolytic cathode can be an uncoated titanium plate.

The method can include treating the aqueous composition with the electrolytic cell for a duration of 1 sec to 24 h, or 50 min to 200 min, or less than or equal to 24 h and greater than or equal to 1 sec and less than, equal to, or greater than 0.5 sec, 1, 2, 4, 6, 10, 20, 30, 40, 50 sec, 1 min, 1.5, 2, 2.5, 3, 4, 5, 10, 15, 20, 30, 40, 50, 60, 70, 80, 90, 100, 110, 120, 130, 140, 150, 160, 170, 180, 190, 200 min, 4 h, 5, 6, 8, 10, 12, 14, 16, 18, 20, or 22 h. The treatment with the electrolytic cell can include applying a voltage across the electrolytic anode and the electrolytic cathode sufficient to generate a current density in the electrolytic cell of 5-500 mA/cm², or 10-40 mA/cm², or less than or equal to 500 mA/cm² and greater than or equal to 5 mA/cm² and less than, equal to, or greater than 10, 15, 20, 25, 30, 35, 40, 45, 50, 55, 60, 65, 70, 80, 90, 100, 120, 140, 160, 180, 200, 220, 240, 260, 280, 300, 350, 400, or 450 mA/cm².

The destroying the one or more PFAS can include treatment with a plurality of the electrolytic cells. The plurality of the electrolytic cells can include 2 to 1,000 of the electrolytic cells, such as 2 of the electrolytic cells, or 3, 4, 5, 6, 7, 8, 9, 10, 11, 12, 13, 14, 15, 16, 17, 18, 19, or 20 or more of the electrolytic cells.

In various aspects, the electrolytic cell can include one or more bipolar plates between the anode and the cathode in a spaced configuration such that there is a gap between the anode and the one or more bipolar plates, a gap between the cathode and the one or more bipolar plates, and a gap between each of the bipolar plates if more than one bipolar plate is present, wherein the gap can have a size of 0.5 mm to 10 mm, or 1 mm to 3 mm, or less than or equal to 10 mm and greater than or equal to 0.5 mm and less than, equal to, or greater than 0.6 mm, 0.8, 1, 1.2, 1.4, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.2, 4.4, 4.6, 4.8, 5, 5.5, 6, 6.5, 7, 7.5, 8, 8.5, 9, or 9.5 mm. The bipolar electrodes can include titanium, stainless steel, or a combination thereof. The one or more bipolar electrodes can include titanium. The one or more bipolar electrodes can be free of coatings, such as catalyst coatings. The one or more bipolar electrodes can include a catalyst coating. The one or more bipolar electrodes can be titanium electrodes with a catalyst coating thereon. The one or more bipolar electrodes can include the catalyst coating on both major faces of bipolar plates. The one or more bipolar electrodes can include the catalyst coating on one major face of each of the bipolar plates (e.g., the major face that faces the cathode) and the other face of each of the bipolar plates (e.g., the major face that faces the anode) can be free of catalyst coatings or can include a catalyst coating having a different composition than the catalyst coating on the other major face. The catalyst coating can be any suitable catalyst coating. The catalyst coating can include one or more transition metal oxides. The catalyst coating can include TiO₂, RuO₂, IrO₂, Cu, Ag, Co, Ni, Fe, Mo, Cu/Co, Cu/Ni, or a combination thereof (e.g., alloys thereof). The catalyst coating can include TiO₂, RuO₂, IrO₂, such as in a ratio of (50-90):(10-50):(0.1-20) or (60-80):(20-30):(1-6) TiO₂: RuO₂: IrO₂. The catalyst coating can include 70:27:3 TiO₂: RuO₂: IrO₂. The catalyst coating can include Cu/Co. The catalyst coating can include Cu/Ni. The bipolar plate can include one coating on the major face that faces the cathode (e.g., TiO₂, RuO₂, IrO₂, or a combination thereof) and a different coating on the major face that faces the anode (e.g., Cu, Ag, Co, Ni, Fe, Mo, or a combination thereof, such as Cu/Co and/or Cu/Ni).

The electrolytic anode, electrolytic cathode, and one or more bipolar electrodes can be a first electrolytic anode, a first electrolytic cathode, and first one or more bipolar electrodes, wherein the destroying the one or more PFAS can further include treatment with a second electrolytic cell including the first electrolytic cathode, one or more second bipolar electrodes, and a second electrolytic anode, wherein the second bipolar electrodes are between the first electrolytic cathode and the second electrolytic anode. The destroying the one or more PFAS can further include treatment with a third electrolytic cell including the second electrolytic anode, one or more third bipolar electrodes, and a second electrolytic cathode.

The electrolytic anode, electrolytic cathode, and bipolar electrodes can be a first electrolytic anode, a first electrolytic cathode, and first bipolar electrodes, wherein the destroying the one or more PFAS can further include treatment with a second electrolytic cell including the first electrolytic anode, one or more second bipolar electrodes, and a second electrolytic cathode, wherein the second bipolar electrodes are between the first electrolytic anode and the second electrolytic cathode. The destroying the one or more PFAS can further include treatment with a third electrolytic cell including the second electrolytic cathode, one or more third bipolar electrodes, and a second electrolytic anode.

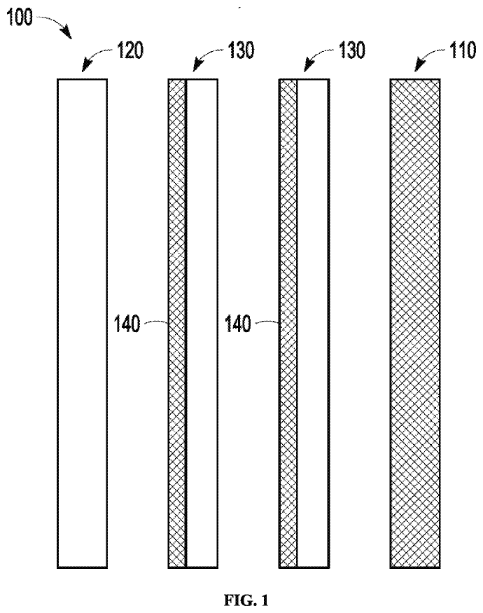

FIG. 1 illustrates an electrolytic cell 100 for destroying the one or more PFAS. The electrolytic cell includes an electrolytic anode plate 110. The electrolytic cell includes an electrolytic cathode plate 120. The electrolytic anode plate includes a catalyst coating. The electrolytic cell includes two bipolar plates 130 that are positioned between the electrolytic anode plate 110 and the electrolytic cathode plate 120. The two bipolar plates 130 each include a catalyst coating on one major face thereof 140 that is facing the electrolytic cathode plate 120.

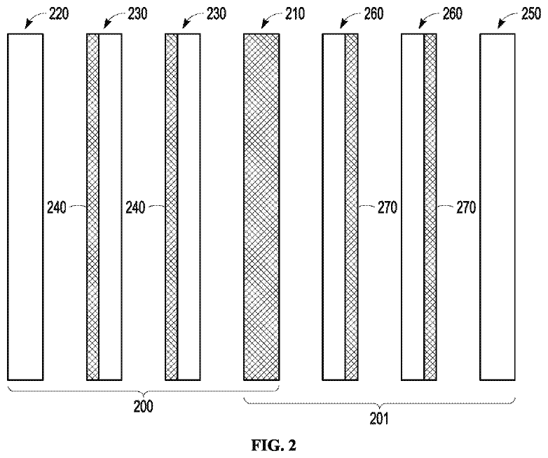

FIG. 2 illustrates two electrolytic cells include a first electrolytic cell 200 and a second electrolytic cell 201 for destroying the one or more PFAS. The first electrolytic cell 200 includes a first electrolytic cathode plate 220, a first electrolytic anode plate 210, and two first bipolar plates 230 positioned between the first electrolytic cathode plate 220 and the first electrolytic anode plate 210. The first electrolytic anode plate 210 includes a catalyst coating. The two first bipolar plates 230 includes a catalyst coating on one major face thereof 240 that is facing the first electrolytic cathode plate 220. The second electrolytic cell 201 includes the first electrolytic anode plate 210, a second electrolytic cathode plate 250, and two second bipolar plates 260. The two second bipolar plates 260 each include a catalyst coating on one major face thereof 270 that is facing the second electrolytic cathode plate 250.

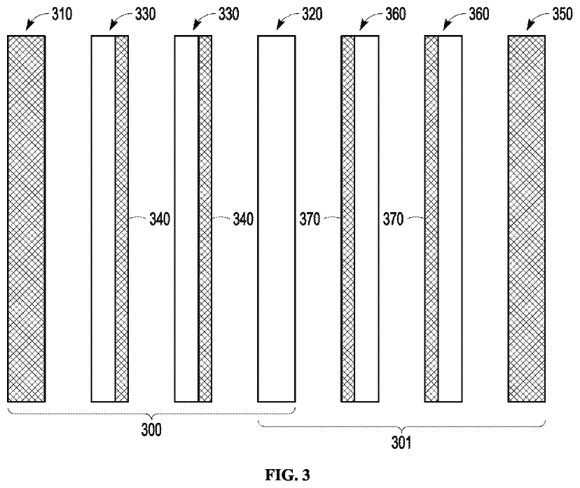

FIG. 3 illustrates two electrolytic cells including a first electrolytic cell 300 and a second electrolytic cell 301 for destroying the one or more PFAS. The first electrolytic cell 300 includes a first electrolytic anode plate 310, a first electrolytic cathode plate 320, and two first bipolar plates 330 positioned between the first anode plate 310 and the first electrolytic cathode plate 320. The first electrolytic anode plate 310 includes a catalyst coating. The two first bipolar plates 330 include a catalyst coating on one major face thereof 340 that is facing the first electrolytic cathode plate 320. The second electrolytic cell 301 includes the first electrolytic cathode plate 320, a second electrolytic anode plate 350, and two second bipolar plates 360. The second bipolar plates 360 are between the first electrolytic cathode plate 320 and the second electrolytic anode plate 350. The second electrolytic anode plate 350 includes a catalyst coating. The second bipolar plates 330 each include a catalyst coating on one major face thereof 370 that is facing the first electrolytic cathode plate 320.

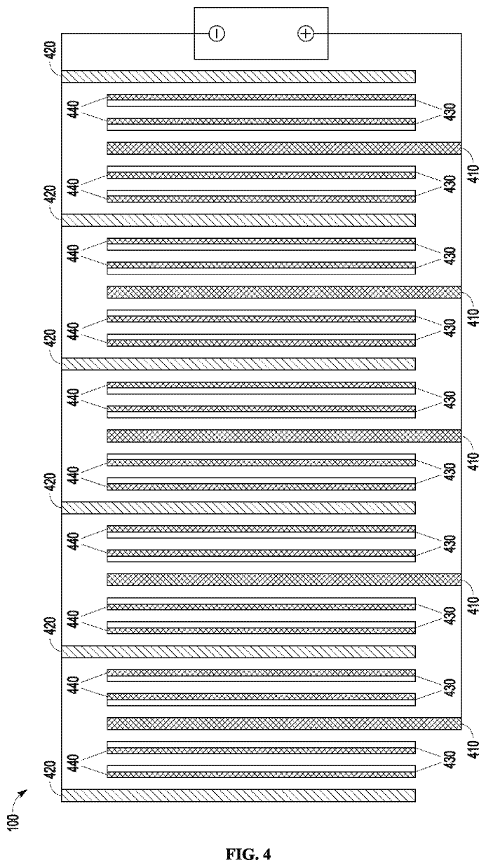

FIG. 4 illustrates a plurality of electrolytic cells 400 for destroying the one or more PFAS. Each electrolytic cell includes an electrolytic anode 410, an electrolytic cathode 420, and two bipolar plates 430. The electrolytic anode 410 includes a catalyst coating. The bipolar plates each include a catalyst coating on a major face thereof 440 that faces the nearest electrolytic cathode 420. FIG. 4 illustrates 10 electrolytic cells.

The aqueous composition that is treated by the electrolytic cell can be formed in any suitable way. In various aspects, the aqueous composition including the one or more PFAS is formed via aeration (e.g., via bubbling of one or more gases therein, such as air, an inert gas, nitrogen, hydrogen, a noble gas, helium argon, xenon, or a combination thereof) of an acidified solution (e.g., having a pH of 0.5 to 5.5, 0.5 to 4, or 1 to 3.5, or 1.5 to 3, or less than or equal to 5.5 and greater than or equal to 0.5 and less than, equal to, or greater than 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 35, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4) to form a foam thereon, and removal of the foam from the acidified solution, wherein the removed foam is the aqueous composition. In various aspects, the aqueous composition can include the metal component, or the aqueous composition can be substantially free of the metal component.

In various aspects, the aqueous composition includes the metal component, and the aqueous composition is formed via treatment of feed water including the one or more PFAS with the metal component, removal and/or concentration of the metal component from the treated feed water, acidification of the removed/concentrated metal component to at least partially dissolve the removed/concentrated metal component and to form an acidified solution (e.g., having a pH of 0.5 to 5.5, 0.5 to 4, or 1 to 3.5, or 1.5 to 3, or less than or equal to 5.5 and greater than or equal to 0.5 and less than, equal to, or greater than 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 35, 3.6, 3.7, 3.8, 3.9, 4, 4.1, 4.2, 4.3, 4.4, 4.5, 4.6, 4.7, 4.8, 4.9, 5, 5.1, 5.2, 5.3, or 5.4), aeration of the acidified solution (e.g., via bubbling of one or more gases therein, such as air, an inert gas, nitrogen, hydrogen, a noble gas, helium argon, xenon, or a combination thereof) to form a foam thereon, and removal of the foam from the acidified solution, wherein the removed foam is the aqueous composition.

In aspects herein wherein a foam is placed in the electrolytic cell, or wherein the aqueous composition is formed from a foam, the bubbles in the foam can be allowed to at least partially or fully dissipate prior to the electrolytic treatment.

The combination of treating an aqueous composition with electrolytic destruction and aeration can form a clarified aqueous composition having a low concentration of the PFAS more efficiently than performing electrolytic destruction alone on the aqueous composition to generate the same low concentration of PFAS. The concentrate formed by the method can then be re-subjected to the electrolytic destruction for further destruction of PFAS. By only performing the electrolytic destruction on an aqueous solution having a significant concentration of PFAS, and by avoiding performing the electrolytic destruction on an aqueous composition with dilute concentrations of PFAS wherein the energy spent on the electrolytic destruction has diminishing returns, the combination of electrolytic destruction and aeration can more efficiently reduce PFAS concentrations as compared to electrolytic destruction alone. Electrolytically destroying the PFAS in the aqueous composition and iteratively electrolytically destroying the PFAS in the concentrate formed in the method can destroy PFAS more efficiently compared to a single step of electrolytic destruction, including because the electrolytic destruction is performed at higher concentrations of PFAS when the electrolytic destruction is more efficient.

Aspects of the present disclosure are not limited by any particular mechanism. Increased electrolytic destruction efficiency because of increased concentration can be driven by Fick's First Law: J=−D dC/dx, wherein J is the flux in mass per area per time, D is the diffusion coefficient and dC/dx is the concentration gradient. This equation can be rewritten as J=kₘ (C_{bulk}−C_{surface}), where kₘ is the diffusion coefficient divided by the boundary layer thickness. This equation holds true when the boundary layer thickness is small as controlled by flow rate and spacing between plates in system described herein. Increasing the concentration of PFAS in the bulk directly therefore relates to increasing the concentration of PFAS at the surface of the electrode and thus increasing breakdown efficiency. At lower concentrations of PFAS, parts of the electrodes are effectively starved of PFAS.

### EXAMPLES

Various aspects of the present disclosure can be better understood by reference to the following Examples which are offered by way of illustration. The present disclosure is not limited to the Examples given herein.

### Example 1. Foam Separation of PFAS

FIG. 5 illustrates an apparatus for performing a method of concentrating a fluoroalkyl compound. Body I refers to a lower portion of a fractionator where the water containing PFAS was initially placed, through which air was passed to concentrate the PFAS in the upper part (Body II) in froth formed by the bubbling air. The bottom water at the lower portion of the fractionation process is called “depleted”. Water traps were used to collect compounds evaporating in air leaving the top of Body II using water with a pH value between 11.0-13.0. In all experiments, treatment plant (Glendale) wastewater was used at pH 2.0, with an initial spike of PFNA, PFOA, PFOS, PFHxS, and PFBS. Masses of PFAS compounds were determined using LC-MS. The fractionation was performed for 1 hour at ambient conditions.

Tables 1 and 2 show the mass of the five studied compounds PFNA, PFOS, PFOA, PFHxS, and PFBS at the beginning (“initial”) and at the end (“froth”, “depleted”, and “water trap”) of the foam fractionation as performed without aluminum ions (Table 1) and with aluminum ions (Table 2, +350 ppm Al). In Tables 1-2, “initial” refers to the initial mass of each of the compounds present in the volume of water to be fractionated, “froth” is the mass of compound collected in the froth (Body II), “depleted” is the mass of compound remaining in the bottom water after the process, “water trap” is the mass of compound in the water traps after the process, and “% removal” is the mass percentage of the PFAS compounds removed from the initial volume of water that was fractionated based on the mass of compounds in the depleted water ((initial-depleted)/initial). The results in Table 1A show that in the fractionation in the absence of aluminum, more than 90% of all compounds are removed from the water except for PFBS with a 42% removal. Table 1B gives the raw concentration data from the LC-MS used to generate the data in Table 1A. In the presence of 350 mg of aluminum in the initial volume of water to be fractionated, the results in Table 2A show a removal of greater than 90% in all cases, including for PFBS. Table 2B gives the raw concentration data from the LC-MS used to generate the data in Table 2A. In both experiments, the mass of compound collected in the water trap was very low, indicating a low volatilization value.

TABLE 1A
Initial, froth, depleted, water trap mass, and percentage
removal of PFAS compounds without addition of aluminum.

| | PFNA | PFOS | PFOA | PFHxS | PFBS |
|---|---|---|---|---|---|
| | (ng) | (ng) | (ng) | (ng) | (ng) |
| Initial | 83 | 267 | 345 | 140 | 812 |
| Froth | 55.1 | 234.5 | 227.9 | 143.9 | 375.8 |
| Depleted | 7 | 7 | 10 | 7 | 471 |
| Water Trap | 0.3 | 0.7 | 2.8 | 0.3 | 0.3 |
| % Removal | 91.6 | 97.4 | 97.1 | 90.18 | 42.0 |

TABLE 1B
Initial, froth, depleted, and water trap concentrations,
without addition of aluminum.

| | PFNA | PFOS | PFOA | PFHxS | PFBS |
|---|---|---|---|---|---|
| | (ppt) | (ppt) | (ppt) | (ppt) | (ppt) |
| Initial | 83 | 267 | 345 | 140 | 812 |
| Froth | 1377 | 5862 | 5697 | 3597 | 9394 |
| Depleted | 7 | 7 | 10 | 7 | 471 |
| Water Trap | 1 | 2 | 8 | 1 | 1 |

TABLE 2A
Initial, froth, depleted, water trap mass, and percentage removal
of PFAS compounds with addition of 350 ppm aluminum.

| | PFNA | PFOS | PFOA | PFHxS | PFBS |
|---|---|---|---|---|---|
| | (ng) | (ng) | (ng) | (ng) | (ng) |
| Initial | 96 | 281 | 276 | 150 | 96 |
| Froth | 36 | 161.1 | 120.15 | 90.18 | 36 |
| Depleted | 7 | 7 | 7 | 7 | 7 |
| Water Trap | 0.06 | 0.35 | 0.175 | 0.035 | 0.06 |
| % Removal | 92.7 | 97.5 | 97.5 | 95.3 | 92.7 |

TABLE 2B
Initial, froth, depleted, and water trap concentrations,
with addition of 350 ppm aluminum.

| | PFNA | PFOS | PFOA | PFHxS | PFBS |
|---|---|---|---|---|---|
| | (ppt) | (ppt) | (ppt) | (ppt) | (ppt) |
| Initial | 96 | 281 | 276 | 150 | 722 |
| Froth | 1200 | 5370 | 4005 | 3006 | 19818 |
| Depleted | 7 | 7 | 7 | 7 | 62 |
| Water Trap | 2 | 1 | 5 | 1 | 1 |

The foam was passed through a pipe and collected in a graduated cylinder. The volume of the initial, froth, depleted, and water trap portions was used to convert the concentration of these portions as measured by LC-MS to mass. Some foam was likely left on the walls at the top of the fractionator, in the pipe, and in the graduated cylinder, which is a possible explanation of why the water trap, depleted, and froth masses total to slightly less than the initial mass.

### Example 2. Foam Separation of PFAS

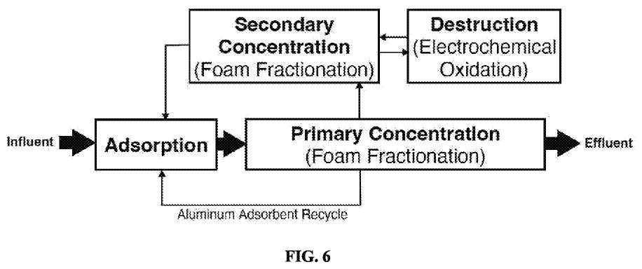

A simplified process flow diagram of the apparatus used for foam separation of PFAS in this Example is shown in FIG. 6. A detailed process flow diagram of the apparatus is shown in FIG. 7. During the adsorption step, added aluminum concentrate (formed via dissolution of aluminum metal in HCl) formed aluminum hydroxide. The added aluminum concentrate had a pH of <2.7. The pH in the adsorption tank was between 4.5 and 5.2, and the adsorption tank had a concentration of aluminum of about 150 ppm. The conditions and retention times of the adsorption step were determined by treatability testing. During the primary concentration and effluent filtration step, sequential foam fractionation of the effluent water from the adsorption step was performed. During the primary concentration and effluent filtration step, the pH of the water was between 4.5 and 5.2. After sequential fractionator 3 and before the aluminum filtration, the water was basified to pH 6-7 via addition of sodium hydroxide. Filtration was used to recover solid aluminum hydroxide from the water. The recovered solid aluminum hydroxide, which included residual effluent water, was acidified to a pH of less than 4 prior to dissolve the majority of the aluminum hydroxide, which was then pumped back to the adsorption step. During the secondary concentration step, a hydrophobic foam fractionation was performed at low pH (greater than or equal to 2.6 to less than 4.0), separating PFAS and aluminum, and concentrating the PFAS for destructive treatment. The residual that remained after foaming in the secondary concentration step was recycled back to the adsorption step, thereby recycling both aluminum and the residual water. During the destruction step, the concentrated PFAS was treated with an electrolytic reactor to cause mineralization and destruction of the PFAS. The adsorption, primary concentration, and secondary concentration steps were performed continuously, while the destruction step was performed in a batch fashion. Concentrations of PFAS compounds were determined using LC-MS (via EPA method 1633 or 537). The TOC was determined via protocol SW846 using method 9060A. Aluminum concentration was measured via inductively coupled plasma (ICP). AOF was measured via combustion ion chromatography (CIC).

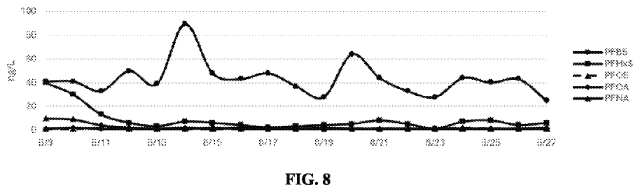

A 26-day continuous flow test at 3 gph (72 gpd) was performed. The influent water was 1,760 gallons with >10,000 ng/L of total PFAS and >60 mg/L total organic carbon (TOC). The PFAS concentrations in the effluent water met maximum concentration limits (MCLs) for 19 consecutive days with a single pass through the system. The primary concentration generated 192 gallons which were further processed in the secondary concentration step. Table 3 illustrates the influent concentration, average effluent concentration, and average % removal for the measured components of the water. FIG. 8 illustrates a graph showing effluent concentrations of various PFAS during the 26-day continuous flow test at 3 gph.

TABLE 3
Influent concentration, average effluent concentration,
and average % removal for the measured components.

| | | Average | |
|---|---|---|---|
| | Influent | effluent | Average % |
| Analyte | concentration | concentration | removal |
| PFBS (ng/L) | 61 | 43 | 29.4% |
| PFHxS (ng/L) | 594 | 9 | 98.6% |
| PFOS (ng/L) | 1,421 | 1 | 99.9% |
| PFOA (ng/L) | 318 | 1 | 99.7% |
| PFNA (ng/L) | 7,138 | 2 | 100.0% |
| TOC (mg/L) | 66 | 26 | 60.6% |
| DRO (mg/L) | 0.26 | 0.17 | 34.6% |
| GRO (mg/L) | 0.58 U | 0.1 | 82.8% |
| AOF (μg/L) | 9.1 J | 1.0 U | 89.0% |
| Aluminum (μg/L) | 49 | 94 | NA |

DRO = diesel range organics.
GRO = gasoline range organics.
AOF = adsorbable organic fluorine.
U = compound was analyzed for but not detected; method detection limit reported.
J = concentration is an approximate value.

A 21-day continuous flow test was conducted at 2 gph (48 gpd). The influent water was 1,500 gallons having >40,000 ng/L of total PFAS and ~25,000 ng/L PFOS. During week 1, the process equipment and conditions were optimized. Aluminum was successfully recycled into the adsorption tank during the 13 days of treatment post optimization. The PFAS were concentrated into <1.2 L (0.32 gallons), a >3,600X volume reduction, from the influent volume of 1,150 gallons with only the lead portion of the secondary concentration completed. An additional 20 to 50-times concentration yielding a total of 100,000×volume reduction could be expected but insufficient volume existed to complete the lag fractionation during this test. The volume reduction suggests that a 20-gpm system (10.5 M gal/yr) achieving 100,000×concentration factor could yield as little as 100 gallons for destruction annually. Table 4 illustrates PFAS concentration of influent and effluent water during the 21-day continuous flow test.

TABLE 4
PFAS concentration of influent and effluent
water during the 21-day continuous flow test.

| | PFAS concentration (ng/L) | | |
|---|---|---|---|
| | | Treated effluent | Average % |
| Analyte | Influent | 13-day average | removal |
| PFBS | 245 | 153 | 37.60% |
| PFHxS | 15,133 | 9 | 99.90% |
| PFOS | 24,964 | 11 | 100.00% |
| PFOA | 1,267 | 1 | 99.90% |
| PFNA | 1,443 | 2 | 99.90% |

A 5-day continuous flow test was conducted at 2 gph (48 gpd). The influent water was 240 gallons having >680,000 ng/L total PFAS and ~480,000 ng/L PFOS. The operating conditions were consistent with those used for the 21-day run. The test resulted in >99.9% removal of all regulated compounds. The test demonstrated that the process is adaptable to influent water having inconsistent or higher PFAS concentrations. This non-optimized test demonstrated that operating costs are not impacted by higher PFAS concentration in influent water. Table 5 illustrates PFAS concentration of influent and effluent water during the 5-day continuous flow test.

TABLE 5
PFAS concentration of influent and effluent
water during the 5-day continuous flow test.

| | PFAS concentration (ng/L) | | |
|---|---|---|---|
| | | Treated effluent | Average % |
| Analyte | Influent | 5-day average | removal |
| PFHxS | 160,000 | 161 | 99.90% |
| PFOS | 480,000 | 36 | 99.99% |
| PFOA | 6,300 | 1 | 99.98% |
| PFNA | 420 U | 1 | — |

U = compound was analyzed for but not detected; method detection limit
reported.

### Example 3. Electrochemical Mineralization and Fractionation

A mixture of PFAS was spiked into municipal wastewater effluent to create a source water for a destruction test using a 1 liter (L) electrochemical cell with a bipolar configuration designed to create a high ratio of the active electrode area to the volume of treated water. The cell was composed of mixed metal oxide (MMO) anodes that were titanium plates coated with titanium oxide/ruthenium-iridium oxide and uncoated titanium plate cathodes. The gap between the electrodes and the bipolar plates, and the gap between the bipolar plates, was 3 mm. The current density used during operation was 45 mA/cm². The configuration of the electrochemical cell corresponded to that shown in FIG. 2. Oxidation of PFAS compounds occurred both by direct reaction at the anode and by reaction with sulfates and OH radicals that were generated electrochemically following the addition of sulfate and persulfate ions into the cell. PFAS mineralization occurred more rapidly at higher PFAS concentrations and asymptotically declined with time as the PFAS remaining available to contact the anode and cathode plates was reduced. The electrochemical cell operated at low pH of 2 to 2.5 that both supported PFAS mineralization kinetics and prevents the generation of chlorate or perchlorate that are common adverse byproducts of destruction technologies. A water trap with high pH of 12 was used to capture any PFAS that were volatilized during operation of the electrochemical cell. Direct current was applied to the cell for 8 hours of operation and post-electrolysis, the content of the electrochemical cell was transferred to a 1 L fractionation column. The pH was increased to 4.5 to produce (i.e., precipitate) aluminum hydroxide particles that adsorb PFAS remaining after electrolysis. Fractionation was completed by passing air for 60 minutes at low flow before the air flow rate was increased for 40 minutes to generate a froth that was removed from the fractionation column. The volume of froth collected was 0.07 L, approximately 0.8% of the volume of the electrochemical cell influent. Samples were collected of the influent for the destruction test, the post-electrolysis water, the generated froth, and the post-fractionation water (i.e., the water from which the froth was removed). Table 6 shows destruction efficiency achieved at the four points sampled in the experiment. The results for the froth sample indicate PFAS remaining post-electrolysis can be effectively reconcentrated into a small volume (0.07 L) that can be further subjected to hydrolysis at a faster rate than the unconcentrated PFAS due to the higher concentrations. The post-fractionation sample demonstrates up to 99.9% removal of individual PFAS remaining in the post electrolysis water resulting in concentrations that enable recycling or further treatment of the water.

TABLE 6
Enhanced destruction efficiency of PFAS by combining electrolysis and fractionation.

| Sample | PFBA | PFBS | PFHxS | PFOS | PFOA | PFNA |
|---|---|---|---|---|---|---|
| Destruction Influent (ng/L) | 4713 | 2979 | 4624 | 30448 | 2839 | 2136 |
| Post-Electrolysis (ng/L) | 1470 | 6054 | 767 | 3665 | 27 | 20 |
| Post-Fractionation (ng/L) | 1136 | 35 | 35 | 26 | 16 | 9 |
| Destruction Removal Efficiency (%) | 75.9 | 98.8 | 99.2 | 99.9 | 99.4 | 99.6 |
| Reconcentrated Froth (ng/L) | 2869 | 61316 | 8258 | 23037 | 3176 | 1083 |

### Example 4. Electrochemical Mineralization and Fractionation

The same electrochemical cell with bipolar configuration as described in Example 3 was tested to evaluate the impact on PFAS concentrations of two different active electrolysis times followed by fractionation. The test influent contained high PFAS concentrations generated by foam fractionating a source groundwater. The foam fractionate influent did not contain aluminum. The active mineralization phase was terminated after 10 hours for the first experiment and after 24 hours for the second experiment, at which time the post-electrolysis liquid was transferred to the same fractionation column used in Example 3. Aluminum was added to achieve a concentration of 150 mg/L and the pH increased to 4.5 to promote the formation of aluminum hydroxide particles and adsorption of PFAS remaining post-electrolysis. Samples were collected for PFAS analysis of the initial influent and post-fractionation liquids that are summarized in Table 7. This test demonstrated that increasing the time of electrolysis resulted in lower PFAS concentrations in the post-fractionation water and confirmed the conclusions of Example 3.

TABLE 7
Impact of electrolysis time on PFAS removal post-fractionation.

| | | 10 Hours | 24-Hour | | |
|---|---|---|---|---|---|
| | | electrol- | electrol- | | |
| | Destruc- | ysis + | ysis + | 10-Hour | 24-Hour |
| PFAS | tion | fraction- | fraction- | PFAS | PFAS |
| (chain | Influent | ation | ation | reduction | reduction |
| length) | (ng/L) | (ng/L) | (ng/L) | (%) | (%) |
| PFBS (4) | 100 U | 570 | 23 | — | — |
| PFBA (4) | 30 U | 2,300 | 1,200 | — | — |
| PFHxS (6) | 46,000 | 660 | 39 | 98.6% | 99.9% |
| PFOS (8) | 490,000 | 360 | 170 | 99.9% | 99.9% |
| 6:2 FTS (8) | 1,800 | 25 | 7 | 98.6% | 99.6% |
| PFOA (8) | 55,000 | 1,000 | 82 | 98.2% | 99.9% |
| PFNA (9) | 20 U | 0.19 U | 0.44 U | — | — |

“U” indicates that the compound was analyzed but not detected at the reported concentration.
“—” indicates that % was not calculated.

### Example 5. Electrochemical Mineralization and Fractionation

A destruction demonstration was completed on 1 liter (0.26 gallons) of PFAS concentrate generated by treating 1760 gallons of groundwater using aluminum as an adsorbent. The destruction test procedure applied a direct current for 16 hours to the same electrochemical cell operating under the test conditions used in Examples 3 and 4. A 16-hour duration was selected based on additional tests that showed that the rate of PFAS mineralization declined after 16 hours on this influent water. The post-electrolysis water was transferred to a 1 L fractionation column and the pH was increased from 2 to 4.5 to promote the formation of aluminum hydroxide particles to adsorb PFAS remaining post-electrolysis. The fractionation was completed by passing air at a low flow rate for 60 minutes before increasing the air flow rate to create a froth that was continuously removed for 40 minutes. The volume of froth generated was 50 mL (0.013 gallons). The concentration of PFAS in the influent groundwater was measured. Samples of the destruction influent, post-electrolysis and fractionation liquid, and reconcentrated froth were collected and analyzed for PFAS. The concentrations are summarized in Table 8. These data demonstrate that the combination of 16 hours of electrolysis followed by fractionation can significantly mineralize PFAS and create a post fractionation water with PFAS concentrations below or near the original influent groundwater to enable further treatment and generate a small volume, high-concentration froth for further destruction.

The electric current and operating potential in the electrochemical cell were constant during electrolysis; therefore, total energy cost is directly associated with the electrolysis time. Extrapolation of the first-order kinetics obtained in the electrochemical experiments indicated that to obtain the concentration values of PFAS compounds after fractionation with only the application of the electrochemical process, a time of about 100 hours would be needed. Therefore, the combination of electrolysis and fractionation results in a reduction of energy costs of about 84% (((100 h-16 h)/100 h)*100) to achieve similar PFAS concentrations to electrolysis alone.

TABLE 8
Demonstration of electrolysis and fractionation
combination within complete treatment process.

| | | Volume (gallons): | | | | | | | | |
|---|---|---|---|---|---|---|---|---|---|---|
| | | | | | | 0.26 gal. | | | | |
| | | 1760 gal. | | 0.4 gal. | | 16 hour | | | | |
| | | Influent | | Destruction | | electrolysis + | | Destruction | 0.013 gal. | |
| | Chain | groundwater | | influent | | fractionation | | removal | Reconcentrated | |
| Analyte | length | (ng/L) | | (ng/L) | | (ng/L) | | efficiency | froth (ng/L) | |
| PFBS | 4 | 43 | | 5,000 | U | 420 | | — | 5,900 | J |
| PFBA | 4 | 3 | U | 10,000 | U | 6,800 | | — | 10,000 | U |
| PFMPA | 4 | 3 | U | 5,000 | U | 27 | | — | 5,000 | U |
| PFPeS | 5 | 3 | U | 5,000 | U | 5 | U | — | 23,000 | |
| PFMBA | 5 | 3 | U | 5,000 | U | 9 | J | — | 5,000 | U |

| | | | | | | | |
|---|---|---|---|---|---|---|---|
| PFPeA | 5 | 35 | 5,000 | U | 14,000 | — | 47,000 |

| | | | | | | | |
|---|---|---|---|---|---|---|---|
| PFHxS | 6 | 460 | 51,000 | 23 | J | 99.95% | 890,000 |

| PFHxA | 6 | 150 | | 30,000 | | 14,000 | 53.33% | 2,600,000 | |
|---|---|---|---|---|---|---|---|---|---|
| 4:2 FTSA | 6 | 5 | U | 5,000 | U | 64 | — | 8,300 | J |

| | | | | | | | | | |
|---|---|---|---|---|---|---|---|---|---|
| PFHpS | 7 | 19 | J | 17,000 | J | 10 | J | 99.94% | 100,000 |
| PFHpA | 7 | 3 | U | 10,000 | J | 10 | J | 99.90% | 790,000 |

| PFOS | 8 | 1,800 | | 1,400,000 | 600 | | 99.96% | 2,400,000 | |
|---|---|---|---|---|---|---|---|---|---|
| 6:2 FTS | 8 | 11 | U | 580,000 | 1,100 | | 99.81% | 26,000,000 | |
| PFOA | 8 | 530 | | 360,000 | 89 | | 99.98% | 1,900,000 | |
| PFNA | 9 | 4,900 | J | 1,900,000 | 620 | J | 99.97% | 2,100,000 | J |

| | | | | | |
|---|---|---|---|---|---|
| Totals | 7,937 | 4,348,000 | 37,772 | 99.13% | 36,864,200 |

“U” indicates the was analyzed but not detected at the reported concentration.
“J” indicates an estimated concentration due to detection below reporting limit or quality control.
“—” indicates that % was not calculated.

### Example 6. Electrochemical Mineralization and Fractionation

A destruction demonstration was completed using 1 liter (0.26 gallons) of PFAS concentrate generated by treating 1150 gallons of groundwater using aluminum as an adsorbent. The destruction test procedure applied a direct current for 16 hours to the same electrochemical cell operating under the test conditions used in Examples 3, 4, and 5. A 16-hour duration for the electrolysis was used and the post-electrolysis water was transferred to a 1 L fractionation column. The pH was increased from 2 to 4.5 to promote the formation of aluminum hydroxide particles to adsorb PFAS remaining post-electrolysis. The fractionation was completed by passing air at a low flow rate for 60 minutes before increasing the air flow rate to create a froth that was continuously removed for 40 minutes. The volume of froth generated was 35 mL (0.009 gallons). The concentration of PFAS in the groundwater was measured. Samples of the destruction influent, post-electrolysis and fractionation liquid, and reconcentrated froth were collected and analyzed for PFAS. The concentrations are summarized in Table 9. These data confirm the conclusions of Example 5 that a combination of 16 hours of electrolysis followed by fractionation can significantly mineralize PFAS and create a post fractionation water with PFAS concentrations below or near the original influent groundwater to enable further treatment and generate a small volume, high-concentration froth for further destruction. The combination of electrolysis and fractionation results in reduced energy costs to achieve similar PFAS concentrations than electrolysis alone.

TABLE 9
Second demonstration of electrolysis and fractionation
combination within complete treatment process.

| | | Volume (gallons): | | | | | | | |
|---|---|---|---|---|---|---|---|---|---|
| | | | | | 0.24 gal. | | | | |
| | | 1,150 gal. | 0.26 gal. | | 16 hour | | | | |
| | Carbon | Influent | Destruction | | electrolysis + | | Destruction | 0.009 gal. | |
| | chain | Groundwater | Influent | | fractionation | | removal | Reconcentrated | |
| Analyte | length | (ng/L) | (ng/L) | | (ng/L) | | efficiency | Froth (ng/L) | |
| PFBS | 4 | 245 | 34,580 | | 77 | J | 99.78% | NR | |
| PFBA | 4 | 30 | 14,870 | | 27,000 | | — | 26,417 | |
| PFPeA | 5 | 276 | 11,667 | U | 14,000 | | — | 555,576 | J |
| PFPeS | 5 | 299 | 291,857 | | 50 | U | 99.98% | 1,886,536 | J |
| PFHxA | 6 | 1,811 | 528,202 | | 180 | J | 99.97% | 14,064,226 | J |
| PFHxS | 6 | 15,133 | 9,729,882 | J | 640 | | 99.99% | 41,471,177 | J |
| PFHpA | 7 | 192 | 164,835 | | 80 | U | 99.95% | 1,486,911 | J |
| PFHpS | 7 | 204 | 519,897 | | 50 | U | 99.99% | 2,218,959 | J |
| PFOS | 8 | 24,964 | 10,179,234 | | 1,500 | | 99.99% | 41,232,677 | |
| PFOA | 8 | 1,297 | 439,339 | | 90 | U | 99.98% | 1,442,415 | |
| 6,2 FTS | 8 | 1,443 | 34,412 | J | 130 | J | 99.62% | 655,275 | |
| PFNA | 9 | 1,443 | 476,667 | | 58 | J | 99.99% | 656,413 | |
| PFDA | 10 | 4 U | 18,449 | J | 50 | U | 99.73% | 7,079 | J |
| PFUn(d)A | 11 | 4 U | 33,837 | J | 50 | U | 99.85% | 17,666 | J |

| | | | | | |
|---|---|---|---|---|---|
| Total (ng/L) | 47,092 | 33,096,133 | 43,878 | 99.87% | 105,721,327 |

“U” indicates the was analyzed but not detected at the reported concentration.
“J” indicates an estimated concentration due to detection below reporting limit or quality control.
“NR” indicates not reported.
“—” indicates that % was not calculated.

### Example 7. Electrochemical Mineralization and Fractionation

A field pilot demonstration of a complete groundwater treatment and destruction process was completed at a PFAS-contaminated groundwater site in Florida. The groundwater treatment occurred over 4 weeks and used aluminum as an adsorbent within the process. Foam fractionate concentrated with PFAS was generated and used for a destruction demonstration influent using the same electrochemical cell and conditions used in Examples 3 through 6. A 16-hour duration for the electrolysis was completed and the post-electrolysis water was transferred to a 1 L fractionation column. The pH increased from 2 to 4.5 to promote the formation of aluminum hydroxide particles to adsorb PFAS remaining post-electrolysis. The fractionation was completed by passing air at a low flow rate for 60 minutes before increasing the air flow rate to create a froth that was continuously removed for 40 minutes. The volume of froth generated was 75 mL. The concentration of PFAS in the groundwater was measured. Samples of the destruction influent, post-electrolysis and fractionation liquid, and reconcentrated froth were collected and analyzed for PFAS. The concentrations are summarized in Table 10. These data confirm the conclusions of Examples 5 and 6 that a combination of 16 hours of electrolysis followed by fractionation can significantly mineralize PFAS and create a post fractionation water with PFAS concentrations below or near the original influent groundwater to enable further treatment and generate a small volume, high-concentration froth for further destruction. The combination of electrolysis and fractionation results in reduced energy costs to achieve similar PFAS concentrations than electrolysis alone.

TABLE 10
Third demonstration of electrolysis and fractionation combination within complete treatment process.

| Sample | PFBA | PFBS | | PFPeA | PFPeS | PFHxA | PFHxS | PFHpA |
|---|---|---|---|---|---|---|---|---|
| Influent Groundwater | 63 | 230 | | 96 | 320 | 210 | 1900 | 49 |
| Destruction Influent | 150 J | 2,300 | J | 940 U | 42,000 | 19,000 | 450,000 | 9,300 |
| Post-electrolysis | 2,300 | 2,500 | | 1,700 | 20,000 | 16,000 | 100,000 J | 1,000 |
| Post-fractionation | 1,600 | 2.3 | U | 70 | 18 | 9.1 J | 150 | 4 J |

| | | | | | | | |
|---|---|---|---|---|---|---|---|
| Destruction Removal Efficiency (%) | — | 99.90% | 92.55% | 99.96% | 99.95% | 99.97% | 99.96% |
| Reconcentrated Froth (ng/L) | 2,900 | 35,000 | 22,000 | 240,000 | 220,000 | 1,000,000 | 14,000 |

| Sample | PFHpS | PFOA | PFOSA | | PFOS | 6:2 FTS | PFNA |
|---|---|---|---|---|---|---|---|
| Influent Groundwater | 81 | 130 | 73 | | 2200 | 48 | 13 |
| Destruction Influent | 17,000 | 16,000 | 410 | | 510,000 | 11,000 | 670 |
| Post-electrolysis | 1,200 | 4,800 | 6 | U | 31,000 | 1,900 | 83 |
| Post-fractionation | 6 J | 56 | 2.3 | U | 210 | 18 J | 3.8 J |
| Destruction Removal Efficiency (%) | 99.96% | 99.65% | 99.44% | | 99.96% | 99.84% | 99.43% |
| Reconcentrated Froth (ng/L) | 14,000 | 53,000 | 63 | U | 330,000 | 17,000 | 970 |

“U” indicates the was analyzed but not detected at the reported concentration.
“J” indicates an estimated concentration due to detection below reporting limit or quality control.
“—” indicates that % was not calculated.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present disclosure. Thus, it should be understood that although the present disclosure has been specifically disclosed by specific aspects and optional features, modification and variation of the concepts herein disclosed may be resorted to by those of ordinary skill in the art, and that such modifications and variations are considered to be within the scope of aspects of the present disclosure.

### Exemplary Aspects

The following exemplary aspects are provided, the numbering of which is not to be construed as designating levels of importance:

Aspect 1 provides a method of destroying one or more perfluoro- or polyfluoro-alkyl substances (PFAS), the method comprising:
performing a destruction procedure on an aqueous composition, wherein the aqueous composition comprises the one or more PFAS, to form a treated aqueous composition, wherein the treated aqueous composition has a concentration of the one or more PFAS that is lower than a concentration of the one or more PFAS in the aqueous composition;
aerating the treated aqueous composition to form a foam thereon; and
separating the foam from the treated aqueous composition to form a concentrate and a clarified aqueous composition, wherein the clarified aqueous composition has a concentration of the one or more PFAS that is lower than a concentration of the one or more PFAS in the concentrate.

Aspect 2 provides the method of claim 1, wherein the destruction procedure comprises thermal treatment, treatment with an electrolytic cell, treatment with a plasma reactor, treatment with supercritical water, combustion treatment, oxidation treatment, chemical treatment, or a combination thereof.

Aspect 3 provides the method of claim 1-2, wherein the destruction procedure comprises electrolytically treating the aqueous composition in an electrochemical cell, wherein the aqueous composition has a pH of 0 to 5.5.

Aspect 4 provides the method of Aspect 3, wherein the electrolytic treatment of the aqueous composition transforms at least some of the one or more PFAS therein into carbon dioxide and fluoride.

Aspect 5 provides the method of any one of Aspects 1-4, wherein the aqueous composition further comprises a metal component.

Aspect 6 provides the method of Aspect 5, wherein the metal comprises Mg, Al, Fe, Zn, Cu, Cd, Cr, Hg, Ni, V, Ce, or a combination thereof.

Aspect 7 provides the method of any one of Aspects 4-6, wherein the metal component comprises Al³⁺.

Aspect 8 provides the method of any one of Aspects 4-7, wherein the metal component comprises AlCl₃, Al(OH)₃, AlPO₄, Al₂(SO₄)₃, or a combination thereof.

Aspect 9 provides the method of any one of Aspects 4-8, wherein the metal component comprises aluminum hydroxide.

Aspect 10 provides the method of any one of Aspects 4-9, wherein the clarified aqueous composition comprises the metal component.

Aspect 11 provides the method of Aspect 10, further comprising reusing the metal component in the clarified aqueous composition and/or a metal from the metal component in the clarified aqueous composition in the metal component in a subsequent iteration of the method.

Aspect 12 provides the method of Aspect 11, wherein the reusing of the metal component and/or the metal therefrom comprises flowing a recycle stream comprising at least part of the clarified aqueous composition or an extract thereof to or upstream of the treated aqueous composition of the subsequent iteration of the method.

Aspect 13 provides the method of any one of Aspects 11-12, wherein the reusing of the metal component and/or the metal therefrom further comprises purging one or more acidification contaminants from the metal component and/or the metal therefrom prior to the reuse of the metal component and/or metal therefrom in the subsequent iteration of the method.

Aspect 14 provides the method of Aspect 13, wherein the purging comprises purging the one or more acidification contaminants from a recycle stream comprising at least part of the clarified aqueous composition or an extract thereof prior to the reuse of the metal component and/or metal therefrom in the subsequent iteration of the method.

Aspect 15 provides the method of any one of Aspects 13-14, wherein the purging comprises separating sulfate, chloride, or a combination thereof, from the metal component and/or metal therefrom.

Aspect 16 provides the method of any one of Aspects 13-15, comprising adding a base to a recycle stream comprising at least part of the clarified aqueous composition or an extract thereof to raise a pH thereof to 3 to 12, filtering out a precipitated salt comprising the one or more acidification contaminants from the recycle stream, and adding the filtered recycle stream to or upstream of the treated aqueous composition.

Aspect 17 provides the method of any one of Aspects 13-16, comprising adding a base to a recycle stream comprising at least part of the clarified aqueous composition or an extract thereof to raise a pH thereof to 3 to 12, and filtering out a precipitated salt comprising the metal component from the recycle stream, and adding the precipitated salt to or upstream of the treated aqueous composition.

Aspect 18 provides the method of any one of Aspects 1-17, further comprising removing fluoride from the clarified aqueous composition.

Aspect 19 provides the method of any one of Aspects 4-18, wherein the aqueous composition has a pH of 0 to 5.5, further comprising raising a pH of the clarified aqueous composition to precipitate the metal component therefrom and to form a pH-adjusted aqueous composition.

Aspect 20 provides the method of Aspect 19, wherein the pH of the pH-adjusted aqueous composition is 3 to 12.

Aspect 21 provides the method of any one of Aspects 19-20, wherein the pH of the pH-adjusted aqueous composition is 6 to 6.5.

Aspect 22 provides the method of any one of Aspects 19-21, further comprising separating the precipitated metal component from the pH-adjusted aqueous composition, to form a separated aqueous composition.

Aspect 23 provides the method of any one of Aspects 1-22, wherein the aerating comprises bubbling one or more gases through the treated aqueous composition.

Aspect 24 provides the method of any one of Aspects 1-23, wherein the aerating comprises aerating with air, an inert gas, oxygen, ozone, nitrogen, hydrogen, a noble gas, helium, argon, xenon, or a combination thereof.

Aspect 25 provides the method of any one of Aspects 1-24, wherein the separating of the foam comprises scraping the foam from the top of the treated aqueous composition, scooping the foam from the top of the treated aqueous composition, sucking the foam from the top of the treated aqueous composition, filtering the foam from the treated aqueous composition, decanting the foam from the treated aqueous composition, or a combination thereof.

Aspect 26 provides the method of any one of Aspects 1-25, wherein the separating of the foam comprises scraping and/or scooping the foam from the top of the treated aqueous composition.

Aspect 27 provides the method of any one of Aspects 1-26, wherein the aerating of the treated aqueous composition forms nanobubbles, microbubbles, or a combination thereof.

Aspect 28 provides the method of any one of Aspects 1-27, wherein the aerating of the treated aqueous composition forms bubbles having a diameter of 1 nm to 10 mm.

Aspect 29 provides the method of any one of Aspects 1-28, wherein the aerating comprises flowing a gas into an aeration apparatus that is submerged in the treated aqueous composition.

Aspect 30 provides the method of Aspect 29, wherein the aeration apparatus comprises a gas sparger, a perforated pipe, a sparging rod, a serrated cone, a diffuser ring, a stone diffuser, or a combination thereof.

Aspect 31 provides the method of any one of Aspects 29-30, wherein the aeration apparatus comprises a stone diffuser.

Aspect 32 provides the method of any one of Aspects 1-31, wherein the aqueous composition comprises water from a natural source, an extract from contaminated soil, an extract from contaminated landfill materials, water contaminated with residual fire-fighting foam, industrial wastewater, a concentrate of any one or any combination thereof, or a combination thereof.

Aspect 33 provides the method of any one of Aspects 1-32, wherein the treated aqueous composition has a pH of 0 to 5.5.

Aspect 34 provides the method of any one of Aspects 1-33, wherein the treated aqueous composition has a pH of 0 to 5.2.

Aspect 35 provides the method of any one of Aspects 1-34, wherein the treated aqueous composition has a pH of 4.5 to 5.2.

Aspect 36 provides the method of any one of Aspects 1-35, further comprising adding one or more acids and/or bases to the treated aqueous composition before or during the aeration to achieve and/or maintain the pH of 0 to 5.5.

Aspect 37 provides the method of Aspect 36, wherein the one or more acids comprise HCl, HBr, HNO₃, H₂SO₄, HClO₄, HClO₃, or a combination thereof.

Aspect 38 provides the method of any one of Aspects 36-37, wherein the one or more acids comprise HCl, H₂SO₄, or a combination thereof.

Aspect 39 provides the method of any one of Aspects 1-38, wherein the method further comprises breaking the foam, to form the concentrate.

Aspect 40 provides the method of any one of Aspects 1-39, wherein the concentrate has a concentration of the one or more PFAS of 0.005 ppt to 500,000 ppm, as measured with the concentrate substantially free of bubbles and foam.

Aspect 41 provides the method of any one of Aspects 1-40, wherein the concentrate has a concentration of the one or more PFAS of 0.005 ppt to 1,000 ppm.

Aspect 42 provides the method of any one of Aspects 1-41, wherein a ratio of a concentration of the one or more PFAS in the treated aqueous composition to a concentration of the one or more PFAS in the concentrate is 1:2 to 1:2,000,000.

Aspect 43 provides the method of any one of Aspects 1-42, wherein a ratio of a concentration of the one or more PFAS in the treated aqueous composition to a concentration of the one or more PFAS in the concentrate is 1:5 to 1:40.

Aspect 44 provides the method of any one of Aspects 1-43, wherein the clarified aqueous composition has a concentration of the one or more PFAS of 0.0001 ppt to 100 ppm.

Aspect 45 provides the method of any one of Aspects 1-44, wherein the clarified aqueous composition has a concentration of the one or more PFAS of 0.0001 ppt to 10 ppt.

Aspect 46 provides the method of any one of Aspects 1-45, wherein the clarified aqueous composition has a concentration of the one or more PFAS of 0.0001 ppt to 4 ppt.

Aspect 47 provides the method of any one of Aspects 1-46, wherein a ratio of a concentration of the one or more PFAS in the clarified aqueous composition to a concentration of the one or more PFAS in the aqueous composition is 1:2 to 1:1000.

Aspect 48 provides the method of any one of Aspects 1-47, wherein a ratio of a concentration of the one or more PFAS in the clarified aqueous composition to a concentration of the one or more PFAS in the aqueous composition is 1:5 to 1:40.

Aspect 49 provides the method of any one of Aspects 1-48, wherein the treated aqueous composition having had the foam removed therefrom is a second aqueous composition, further comprising bubbling the gas into the second aqueous composition to form a second foam and removing the second foam from the second aqueous composition, and combining the foam removed from the treated aqueous composition and the second foam to form the concentrate comprising the one or more PFAS, and wherein the second aqueous composition having the second foam removed therefrom is the clarified aqueous composition.

Aspect 50 provides the method of Aspect 49, wherein the second aqueous composition having had the second foam removed therefrom is a third aqueous composition, further comprising bubbling the gas into the third aqueous composition to form a third foam and removing the third foam from the third aqueous composition, and combining the foam removed from the treated aqueous composition, the second foam, and the third foam to form the concentrate comprising the one or more PFAS and wherein the third aqueous composition having the foam removed therefrom is the clarified aqueous composition.

Aspect 51 provides the method of Aspect 50, further comprising repeating sequential foaming of the treated aqueous composition more than two times, and combining the removed foams to form the concentrate comprising the one or more PFAS, and wherein the sequentially foamed aqueous composition having the foams removed therefrom is the clarified aqueous composition.

Aspect 52 provides the method of any one of Aspects 1-51, further comprising performing the aerating of the treated aqueous composition comprising the one or more PFAS to form the foam comprising the one or more PFAS and the removing of the foam from the treated aqueous composition to form the concentrate comprising the one or more PFAS and to form a clarified aqueous composition two or more times using the concentrate as the treated aqueous composition in each iteration of the two or more times.

Aspect 53 provides the method of Aspect 52, further comprising recycling the clarified aqueous composition formed from the second or higher iteration of the two or more times back to or upstream of the treated aqueous composition used in the first foaming step of the method.

Aspect 54 provides the method of Aspect 52-53, wherein during a second or higher iteration of the two or more times, a pH range of the treated aqueous composition is 0 to 4.

Aspect 55 provides the method of any one of Aspects 52-54, wherein during a second or higher iteration of the two or more times, a pH range of the treated aqueous composition is 2.5 to 4.

Aspect 56 provides the method of any one of Aspects 52-55, wherein during a second or higher iteration of the two or more times, the treated aqueous composition having had the foam removed therefrom is a second aqueous composition, further comprising bubbling the gas into the second aqueous composition to form a second foam and removing the second foam from the second aqueous composition, and combining the foam removed from the treated aqueous composition and the second foam to form the concentrate comprising the one or more PFAS, and wherein the second aqueous composition having the second foam removed therefrom is the clarified aqueous composition.

Aspect 57 provides the method of Aspect 56, wherein during the second or higher iteration of the two or more times, the second aqueous composition having had the second foam removed therefrom is a third aqueous composition, further comprising bubbling the gas into the third aqueous composition to form a third foam and removing the third foam from the third aqueous composition, and combining the foam removed from the treated aqueous composition, the second foam, and the third foam to form the concentrate comprising the one or more PFAS and wherein the third aqueous composition having the foam removed therefrom is the clarified aqueous composition.

Aspect 58 provides the method of Aspect 57, during the second or higher iteration of the two or more times, further comprising repeating sequential foaming of the treated aqueous composition more than two times, and combining the removed foams to form the concentrate comprising the one or more PFAS, and wherein the sequentially foamed aqueous composition having the foams removed therefrom is the clarified aqueous composition.

Aspect 59 provides the method of any one of Aspects 52-58, further comprising recycling the clarified aqueous composition formed from the second or higher iteration of the two or more times back to or upstream of the treated aqueous composition used in the first foaming step of the method.

Aspect 60 provides the method of any one of Aspects 52-59, comprising performing the bubbling of the gas into the treated aqueous composition comprising the one or more PFAS to form the foam comprising the one or more PFAS and the removing of the foam from the treated aqueous composition to form the concentrate comprising the one or more PFAS and to form the clarified aqueous composition one or more additional times using the concentrate formed by combining the foam and any additional foams as the treated aqueous composition in each of the one or more additional times.

Aspect 61 provides the method of any one of Aspects 52-60, comprising recycling the clarified aqueous composition formed from performing the bubbling of the gas into the treated aqueous composition comprising the one or more PFAS to form the foam comprising the one or more PFAS and the removing of the foam from the treated aqueous composition to form the concentrate comprising the one or more PFAS and to form the clarified aqueous composition the one or more additional times back to or upstream of the treated aqueous composition used in the first foaming step of the method.

Aspect 62 provides the method of Aspect 52, wherein a ratio of a concentration of the one or more PFAS in the treated aqueous composition used in the first iteration of the two or more times to a concentration of the one or more PFAS in the concentrate formed by the last iteration of the two or more times is 1:4 to 1:100,000.

Aspect 63 provides the method of any one of Aspects 52-62, wherein a ratio of a concentration of the one or more PFAS in the treated aqueous composition used in the first iteration of the two or more times to a concentration of the one or more PFAS in the concentrate formed by the last iteration of the two or more times is 1:10 to 1:80.

Aspect 64 provides the method of any one of Aspects 1-63, wherein the treated aqueous composition having had the foam removed therefrom is a second aqueous composition, further comprising
bubbling the gas into the second aqueous composition to form a second foam and removing the second foam from the second aqueous composition, and combining the foam removed from the treated aqueous composition and the second foam to form the concentrate comprising the one or more PFAS, and wherein the second aqueous composition having the second foam removed therefrom is the clarified aqueous composition;
performing the bubbling of the gas into the treated aqueous composition comprising the one or more PFAS to form the form comprising the one or more PFAS and the removing of the foam from the treated aqueous composition to form the concentrate comprising the one or more PFAS and to form a clarified aqueous composition two or more times using the concentrate as the treated aqueous composition in each iteration of the two or more times, wherein during a second or higher iteration of the two or more times a pH range of the aqueous composition is 0 to less than 4;
wherein during a second or higher iteration of the two or more times, the treated aqueous composition having had the foam removed therefrom is a secondary second aqueous composition, further comprising bubbling the gas into the secondary second aqueous composition to form a secondary second foam and removing the secondary second foam from the secondary second aqueous composition, and combining the foam removed from the treated aqueous composition in the first iteration of the two or more times and the secondary second foam to form the concentrate comprising the one or more PFAS, and wherein the secondary second aqueous composition having the secondary second foam removed therefrom is a secondary clarified aqueous composition; and
recycling the secondary clarified aqueous composition formed from the second or higher iteration of the two or more times back to or upstream of the treated aqueous composition used in the first foaming step of the method.

Aspect 65 provides the method of any one of Aspects 1-64, wherein the aqueous composition has a pH of 0 to 5.5.

Aspect 66 provides the method of any one of Aspects 1-65, wherein the aqueous composition has a pH of 1 to 5.2.

Aspect 67 provides the method of any one of Aspects 1-66, wherein the treated aqueous composition has a pH of 0 to 5.5, or 0 to 5.2, or 4.5 to 5.2.

Aspect 68 provides the method of any one of Aspects 1-67, wherein the one or more PFAS comprise a perfluoroalkyl or polyfluoroalkyl substance (PFAS), perfluoroalkyl substance, a polyfluoroalkyl substance, a perfluoroalkyl acid (PFAA), or a combination thereof.

Aspect 69 provides the method of any one of Aspects 1-68, wherein the one or more PFAS comprise perfluorooctanesulfonic acid (PFOA), perfluorooctyl sulfonate (PFOS), perfluorohexanesulfonic acid (PFHxS), perfluorononanoic acid (PFNA), perfluorobutanesulfonic acid (PFBS), 2-(N-methyl-perfluorooctane sulfonamido) acetic acid, perfluoroheptanoic acid (PFHpA), n-perfluorooctane sulfonic acid, perfluoromethylheptane sulfonic acid, n-perfluorooctanoic acid, a branched perfluorooctanoic acid, perfluorodecanoic acid (PFDA), perfluoroundecanoic acid (PFUn(d)A), perfluorododecanoic acid, 6:2 fluorotelomer sulfonic acid (6:2 FTS), 2,2,3,3-tetrafluoro-3-(trifluoromethoxy) propanoic acid (PFMPA), perfluoropentanesulfonic acid (PFPeS), perfluoro-4-methoxybutanoic acid (PFMBA), perfluoropentanoic acid (PFPeA), perfluorohexanoic acid (PFHxA), 4:2 fluorotelomer sulfonic acid (4:2 FTSA), perfluoroheptanesulfonic acid (PFHpS), perfluorooctanesulfonamide (PFOSA), or a combination thereof.

Aspect 70 provides the method of any one of Aspects 1-69, further comprising adding an additive to the aqueous composition prior to entry of the aqueous composition into the electrolytic cell or while the aqueous composition is in the electrolytic cell, the additive comprising a radical precursor, hydrogen peroxide, a sulfate salt, a sulfite salt, sodium hypochlorite, potassium hypochlorite, NaCl, CaCl₂, KCl, or a combination thereof.

Aspect 71 provides the method of Aspect 70, wherein the radical precursor comprises HOOH, O₃, S₂O₈⁻, I⁻, CO₃²⁻, HCO₃⁻, H₂PO₄⁻, HPO₄²⁻, PO₄³⁻, HSO₅⁻, or a combination thereof.

Aspect 72 provides the method of any one of Aspects 70-71, wherein the additive comprises a sulfate salt, a persulfate salt, or a combination thereof.

Aspect 73 provides the method of any one of Aspects 70-72, wherein the additive comprises sodium sulfate, potassium sulfate, sodium persulfate, potassium persulfate, or a combination thereof.

Aspect 74 provides the method of any one of Aspects 1-73, wherein the clarified aqueous composition has a concentration of the one or more PFAS of 0.000001 ppt (e.g., undetectable) to 45,000 ppm.

Aspect 75 provides the method of any one of Aspects 1-74, wherein the clarified aqueous composition has a concentration of the one or more PFAS of 0.01 ppt (e.g., undetectable) to 10,000 ppm.

Aspect 76 provides the method of any one of Aspects 1-75, wherein the treated aqueous composition has a concentration of the one or more PFAS that is 0.0001% to 90% of a concentration of the one or more PFAS in the aqueous composition.

Aspect 77 provides the method of any one of Aspects 1-76, wherein the treated aqueous composition has a concentration of the one or more PFAS that is 1% to 30% of a concentration of the one or more PFAS in the aqueous composition.

Aspect 78 provides the method of any one of Aspects 1-77, wherein the clarified aqueous composition has a concentration of the one or more PFAS that is 0.0001% to 50% of a concentration of the one or more PFAS in the separated foam.

Aspect 79 provides the method of any one of Aspects 1-78, wherein the clarified aqueous composition has a concentration of the one or more PFAS that is 0.001% to 10% of a concentration of the one or more PFAS in the separated foam.

Aspect 80 provides the method of any one of Aspects 1-79, wherein the aqueous composition has a concentration of the one or more PFAS of 14 parts per trillion (ppt) or higher.

Aspect 81 provides the method of any one of Aspects 1-80, wherein the aqueous composition has a concentration of the one or more PFAS of 4 ppt or higher.

Aspect 82 provides the method of any one of Aspects 1-81, wherein the aqueous composition has a concentration of the one or more PFAS of 1 part per trillion (ppt) to 100,000 parts per million (ppm).

Aspect 83 provides the method of any one of Aspects 1-82, wherein the aqueous composition has a concentration of the one or more PFAS of 20 ppt to 1 ppm.

Aspect 84 provides the method of any one of Aspects 13-83, further comprising removing the treated aqueous composition from the electrolytic cell prior to performing the aerating and the foam separation.

Aspect 85 provides the method of any one of Aspects 3-84, further comprising placing the separated foam back into the electrolytic cell for further electrolytic treatment.

Aspect 86 provides the method of any one of Aspects 3-85, further comprising electrolytically treating the separated foam in the electrochemical cell to form a second treated aqueous composition that has a concentration of the one or more PFAS that is 0.0001% to 90% of a concentration of the one or more PFAS in the separated foam.

Aspect 87 provides the method of any one of Aspects 3-86, comprising repeating the electrolytic treatment, aeration, separation of foam, and placing the separated foam back into the electrolytic cell for further electrolytic treatment three or more times.

Aspect 88 provides the method of any one of Aspects 3-87, further comprising combining the separated foam with the aqueous composition, electrolytically treating the combined separated foam and aqueous composition, and reperforming the aeration and the foam separation.

Aspect 89 provides the method of any one of Aspects 3-88, further comprising monitoring the concentration of the one or more PFAS in the aqueous composition during the electrolytic treatment of the aqueous composition.

Aspect 90 provides the method of Aspect 89, further comprising performing the electrolytic treatment until the concentration of PFAS in the aqueous composition reaches a predetermined concentration.

Aspect 91 provides the method of Aspect 90, wherein the predetermined concentration is 0.0001% to 90% of a starting concentration of PFAS in the aqueous composition.

Aspect 92 provides the method of any one of Aspects 90-91, wherein the predetermined concentration is 1% to 30% of a starting concentration of PFAS in the aqueous composition.

Aspect 93 provides the method of any one of Aspects 89-92, wherein the method comprises using a trap comprising water in the headspace of the electrolytic cell, wherein the monitoring comprises measuring a concentration of the one or more PFAS in the water of the water-filled trap.

Aspect 94 provides the method of Aspect 93, wherein the measuring comprises ion chromatography.

Aspect 95 provides the method of any one of Aspects 3-94, wherein the electrolytic cell comprises an electrolytic anode and an electrolytic cathode.

Aspect 96 provides the method of Aspect 95, wherein the electrolytic anode comprises an anode material comprising a metal oxide, a transition metal oxide, a mixed metal oxide (MMO), Ti₄O₇, PbO₂, boron-doped diamond (BDD), SnO₂, Bi₂O₃, RuO₂, IrO₂, TiO₂, Ta₂O₅, a precious metal, platinum (e.g., platinum coating on titanium), PtO₂, MnO₂, CeO₂, Rh₂O₃, carbon (e.g., BDD, graphite, graphene, or a combination thereof), or a combination thereof.

Aspect 97 provides the method of any one of Aspects 95-96, wherein the electrolytic anode comprises a titanium plate coated with titanium oxide/ruthenium-iridium oxide.

Aspect 98 provides the method of any one of Aspects 95-97, wherein the electrolytic cathode comprises stainless steel, titanium, carbon (e.g., BDD, graphite, graphene, or a combination thereof), carbon steel, a precious metal, platinum, nickel, iron, copper, silver, or a combination thereof.

Aspect 99 provides the method of any one of Aspects 95-98, wherein the electrolytic cathode comprises an uncoated titanium plate.

Aspect 100 provides the method of any one of Aspects 95-99, wherein the treatment with the electrolytic cell comprises applying a voltage across the electrolytic anode and the electrolytic cathode sufficient to generate a current density of 5-500 mA/cm².

Aspect 101 provides the method of any one of Aspects 95-100, wherein the voltage is sufficient to generate a current density of 10-100 mA/cm².

Aspect 102 provides the method of any one of Aspects 3-101, wherein the aqueous composition is formed via aeration of an acidified solution comprising the one or more PFAS to form a foam thereon, and removal of the foam from the acidified solution, wherein the removed foam is the aqueous composition.

Aspect 103 provides the method of any one of Aspects 5-102, wherein the aqueous composition is formed via aeration of an acidified solution comprising the metal component and the one or more PFAS to form a foam thereon, and removal of the foam from the acidified solution, wherein the removed foam is the aqueous composition.

Aspect 104 provides the method of any one of Aspects 2-103, wherein the aqueous composition is formed via treatment of feed water comprising the one or more PFAS with the metal component, removal of the metal component from the treated feed water, acidification of the removed metal component to at least partially dissolve the removed metal component and to form an acidified solution, aeration of the acidified solution to form a foam thereon, and removal of the foam from the acidified solution, wherein the removed foam is the aqueous composition.

Aspect 105 provides a method of destroying one or more perfluoro- or polyfluoro-alkyl substances (PFAS), the method comprising:
electrolytically treating an aqueous composition in an electrochemical cell, wherein the aqueous composition has a pH of 0 to 5.5 and comprises the one or more PFAS, to form an electrolytically-treated aqueous composition, wherein the electrolytically-treated aqueous composition has a concentration of the one or more PFAS that is lower than a concentration of the one or more PFAS in the aqueous composition;
aerating the electrolytically-treated aqueous composition to form a foam thereon; and
separating the foam from the electrolytically-treated aqueous composition to form a concentrate, wherein the electrolytically-treated aqueous composition separated from the foam has a concentration of the one or more PFAS that is lower than a concentration of the one or more PFAS in the separated foam.

Aspect 106 provides a method of destroying one or more perfluoro- or polyfluoro-alkyl substances (PFAS), the method comprising:
electrolytically treating an aqueous composition in an electrochemical cell, wherein the aqueous composition has a pH of 0 to 5.5 and comprises aluminum hydroxide and the one or more PFAS, to form an electrolytically-treated aqueous composition, wherein the electrolytically-treated aqueous composition has a concentration of the one or more PFAS that is 0.0001% to 90% of a concentration of the one or more PFAS in the aqueous composition;
aerating the electrolytically-treated aqueous composition to form a foam thereon;
separating the foam from the electrolytically-treated aqueous composition, wherein the electrolytically-treated aqueous composition separated from the foam has a concentration of the one or more PFAS that is 0.0001% to 50% of a concentration of the one or more PFAS in the separated foam; and
electrolytically treating the separated foam in the electrochemical cell to form a second electrolytically-treated aqueous composition that has a concentration of the one or more PFAS that is 0.0001% to 90% of a concentration of the one or more PFAS in the separated foam.

Aspect 107 provides a method of destroying one or more perfluoro- or polyfluoro-alkyl substances (PFAS), the method comprising:
electrolytically treating an aqueous composition in an electrochemical cell, wherein the aqueous composition has a pH of 0 to 5.5 and comprises aluminum hydroxide and the one or more PFAS, and monitoring the concentration of the one or more PFAS in the aqueous composition during the electrolytic treatment of the aqueous composition and continuing to perform the electrolytic treatment until the concentration of the one or more PFAS in the aqueous composition reaches a predetermined concentration, to form an electrolytically-treated aqueous composition, wherein the electrolytically-treated aqueous composition has a concentration of the one or more PFAS that is 0.0001% to 90% of a concentration of the one or more PFAS in the aqueous composition;
aerating the electrolytically-treated aqueous composition to form a foam thereon; and
separating the foam from the electrolytically-treated aqueous composition, wherein the electrolytically-treated aqueous composition separated from the foam has a concentration of the one or more PFAS that is 0.0001% to 50% of a concentration of the one or more PFAS in the separated foam.

Aspect 108 provides a method of destroying one or more perfluoro- or polyfluoro-alkyl substances (PFAS), the method comprising:
performing a destruction procedure on an aqueous composition, wherein the aqueous composition comprises the one or more PFAS, to form a treated aqueous composition, wherein the aqueous composition comprises an additive comprising a metal component that comprises a metal, wherein the treated aqueous composition has a concentration of the one or more PFAS that is lower than a concentration of the one or more PFAS in the aqueous composition;
aerating the treated aqueous composition to form a foam thereon;
removing the foam from the treated aqueous composition to form a concentrate comprising the fluoroalkyl compound and to form a clarified aqueous composition comprising the metal of the metal component of the additive, wherein a ratio of a concentration of the fluoroalkyl compound in the aqueous composition to a concentration of the fluoroalkyl compound in the concentrate is 1:2 to 1:2,000,000;
performing the destruction procedure on the concentrate; and
reusing the metal from the clarified aqueous composition or an extract thereof, the reusing comprising adding the metal to the treated aqueous composition of a subsequent iteration of the method.

Aspect 109 provides a method of destroying one or more perfluoro- or polyfluoro-alkyl substances (PFAS), the method comprising:
performing a destruction procedure on an aqueous composition, wherein the aqueous composition comprises the one or more PFAS, to form a treated aqueous composition, wherein the aqueous composition has a pH of 0 to 5.5 and comprises an additive comprising a metal component that comprises aluminum, wherein the treated aqueous composition has a concentration of the one or more PFAS that is lower than a concentration of the one or more PFAS in the aqueous composition;
aerating the treated aqueous composition to form a foam thereon;
removing the foam from the aqueous composition, wherein the aqueous composition having had the foam removed therefrom is a second aqueous composition;
aerating the second aqueous composition to form a second foam and removing the second foam from the second aqueous composition, and combining the foam removed from the aqueous composition and the second foam to form a concentrate comprising the fluoroalkyl compound, and wherein the second aqueous composition having the second foam removed therefrom is a clarified aqueous composition comprising the aluminum of the metal component;
reusing the aluminum in the clarified aqueous composition or an extract thereof, the reusing comprising adding the aluminum to the treated aqueous composition;
aerating the concentrate to form a secondary foam comprising the fluoroalkyl compound then removing the secondary foam from the concentrate to form a secondary concentrate comprising the fluoroalkyl compound and to form a secondary clarified aqueous composition, wherein during the formation of the secondary foam the concentrate has a pH of 0 to less than 4, wherein a ratio of a concentration of the fluoroalkyl compound in the treated aqueous composition to a concentration of the fluoroalkyl compound in the secondary concentrate is 1:2 to 1:2,000,000;
performing the destruction procedure on the secondary concentrate; and
recycling the secondary clarified aqueous composition back to or upstream of the treated aqueous composition.

Aspect 110 provides the method of any one or any combination of Aspects 1-109 optionally configured such that all elements or options recited are available to use or select from.

What is claimed is:

1. A method of destroying one or more perfluoro- or polyfluoro-alkyl substances (PFAS), the method comprising:
performing a destruction procedure on an aqueous composition, wherein the aqueous composition comprises the one or more PFAS, to form a treated aqueous composition, wherein the treated aqueous composition has a concentration of the one or more PFAS that is lower than a concentration of the one or more PFAS in the aqueous composition;
aerating the treated aqueous composition to form a foam thereon; and
separating the foam from the treated aqueous composition to form a concentrate and a clarified aqueous composition, wherein the clarified aqueous composition has a concentration of the one or more PFAS that is lower than a concentration of the one or more PFAS in the concentrate.

2. The method of claim 1, further comprising performing the destruction procedure on the concentrate.

3. The method of claim 1, wherein the destruction procedure comprises thermal treatment, treatment with an electrolytic cell, treatment with a plasma reactor, treatment with supercritical water, combustion treatment, oxidation treatment, chemical treatment, or a combination thereof.

4. The method of claim 1, wherein the destruction procedure comprises electrolytically treating the aqueous composition in an electrochemical cell, wherein the aqueous composition has a pH of 0 to 5.5.

5. The method of claim 4, wherein the aqueous composition has a pH of 1 to 3.5.

6. The method of claim 4, wherein the electrolytic cell comprises an electrolytic anode and an electrolytic cathode, wherein the electrolytic anode comprises an anode material comprising a metal oxide, a transition metal oxide, a mixed metal oxide (MMO), Ti₄O₇, PbO₂, boron-doped diamond (BDD), SnO₂, Bi₂O₃, RuO₂, IrO₂, TiO₂, Ta₂O₅, a precious metal, platinum (e.g., platinum coating on titanium), PtO₂, MnO₂, CeO₂, Rh₂O₃, carbon (e.g., BDD, graphite, graphene, or a combination thereof), or a combination thereof, and wherein the electrolytic cathode comprises stainless steel, titanium, carbon (e.g., BDD, graphite, graphene, or a combination thereof), carbon steel, a precious metal, platinum, nickel, iron, copper, silver, or a combination thereof.

7. The method of claim 6, wherein the electrolytic anode comprises a titanium plate coated with titanium oxide/ruthenium-iridium oxide, and wherein the electrolytic cathode comprises an uncoated titanium plate.

8. The method of claim 1, wherein during the aeration the treated aqueous composition has a pH of 4.5 to 5.3.

9. The method of claim 1, wherein the treated aqueous composition further comprises a metal component, wherein the metal component comprises a metal, wherein the metal component comprises an ion of the metal, a solid or dissolved compound of the metal, an elemental form of the metal, or a combination thereof.

10. The method of claim 9, wherein the metal component comprises aluminum hydroxide.

11. The method of claim 9, further comprising raising a pH of the clarified aqueous composition to precipitate the metal component therefrom and to form a pH-adjusted aqueous composition, wherein the pH of the pH-adjusted aqueous composition is 4 to 12, further comprising separating the precipitated metal component from the pH-adjusted aqueous composition, to form a separated aqueous composition.

12. The method of claim 1, wherein the one or more PFAS comprise perfluorooctanesulfonic acid (PFOA), perfluorooctyl sulfonate (PFOS), perfluorohexanesulfonic acid (PFHxS), perfluorononanoic acid (PFNA), perfluorobutanesulfonic acid (PFBS), 2-(N-methyl-perfluorooctane sulfonamido) acetic acid, perfluoroheptanoic acid (PFHpA), n-perfluorooctane sulfonic acid, perfluoromethylheptane sulfonic acid, n-perfluorooctanoic acid, a branched perfluorooctanoic acid, perfluorodecanoic acid (PFDA), perfluoroundecanoic acid (PFUn(d)A), perfluorododecanoic acid, 6:2 fluorotelomer sulfonic acid (6:2 FTS), 2,2,3,3-tetrafluoro-3-(trifluoromethoxy) propanoic acid (PFMPA), perfluoropentanesulfonic acid (PFPeS), perfluoro-4-methoxybutanoic acid (PFMBA), perfluoropentanoic acid (PFPeA), perfluorohexanoic acid (PFHxA), 4:2 fluorotelomer sulfonic acid (4:2 FTSA), perfluoroheptanesulfonic acid (PFHpS), perfluorooctanesulfonamide (PFOSA), or a combination thereof.

13. The method of claim 1, further comprising adding an additive to the aqueous composition prior to entry of the aqueous composition into the destruction process or while the aqueous composition is in the destruction process, the additive comprising a radical precursor, hydrogen peroxide, a sulfate salt, a sulfite salt, sodium hypochlorite, potassium hypochlorite, NaCl, CaCl₂), KCl, or a combination thereof.

14. The method of claim 13, wherein the additive comprises a sulfate salt, a persulfate salt, or a combination thereof.

15. The method of claim 1, wherein the aqueous composition has a concentration of the one or more PFAS of 1 part per trillion (ppt) to 100,000 parts per million (ppm).

16. The method of claim 1, wherein the treated aqueous composition has a concentration of the one or more PFAS that is 0.0001% to 90% of a concentration of the one or more PFAS in the aqueous composition.

17. The method of claim 1, wherein the clarified aqueous composition has a concentration of the one or more PFAS that is 0.0001% to 50% of a concentration of the one or more PFAS in the separated foam.

18. The method of claim 1, further comprising monitoring the concentration of the one or more PFAS in the aqueous composition during the destruction process of the aqueous composition, further comprising performing the destruction process until the concentration of PFAS in the aqueous composition reaches a predetermined concentration that is 0.0001% to 90% of a starting concentration of PFAS in the aqueous composition.

19. The method of claim 1, wherein the aqueous composition is formed via aeration of an acidified solution comprising the one or more PFAS to form a foam thereon, and removal of the foam from the acidified solution, wherein the removed foam is the aqueous composition.

20. A method of destroying one or more perfluoro- or polyfluoro-alkyl substances (PFAS), the method comprising:
electrolytically treating an aqueous composition in an electrochemical cell, wherein the aqueous composition has a pH of 1 to 3.5 and comprises the one or more PFAS, to form an electrolytically-treated aqueous composition, wherein the electrolytically-treated aqueous composition has a concentration of the one or more PFAS that is lower than a concentration of the one or more PFAS in the aqueous composition;
aerating the electrolytically-treated aqueous composition to form a foam thereon;
separating the foam from the electrolytically-treated aqueous composition to form a concentrate and a clarified aqueous composition, wherein the clarified aqueous composition has a concentration of the one or more PFAS that is lower than a concentration of the one or more PFAS in the concentrate; and
electrolytically treating the concentrate in the electrochemical cell.
